(19) United States
(12) Patent Application Publication
(10) Pub. No.: US 2005/0227357 A1
(43) Pub. Date: Oct. 13, 2005

(54) Recombinant Penicillium funiculosum for homologous and heterologous protein production

(75) Inventors: Ralph Bohlmann, Lyon (FR); Nigel Belshaw, Norwich (GB); David Archer, Nuttingham (GB); Marcos Alcocer, Norwich (GB); Neville Marshall Fish, Stockport (GB); Jerome Pierrard, Lyon (FR); Carole Guitton, Lyon (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignees: Aventis Animal Nutrition S.A.; Rhodia Chimie

(21) Appl. No.: 10/947,168

(22) Filed: Sep. 23, 2004

Related U.S. Application Data
(63) Continuation of application No. 09/959,763, filed on Apr. 5, 2002, now abandoned.

(30) Foreign Application Priority Data
May 6, 1999 (WO) PCT/IB99/00856

(51) Int. Cl. C12N 1/16; C12N 15/74

(52) U.S. Cl. 435/484; 435/254.5

(57) ABSTRACT

The present invention relates to a recombinant P. funiculosum strain for the production of homologous and heterologous proteins, and a transformation method for obtaining such recombinant P. funiculosum. Expression cassettes designed with genetic regulatory elements functional in P. funiculosum such as promoters, terminators and signal peptide encoding sequences are also encompassed by the present invention.

The present invention relates to a recombinant Penicillium funiculosum strain for the production of homologous or heterologous proteins, and a transformation method for obtaining such recombinant P. funiculosum. In addition, functional cassettes for expression of homologous and heterologous genes are also encompassed by the present invention.

Micro-organisms are known as producers of a broad spectrum of extracellular enzymes useful in a variety of industrial applications, such as in the baking industry, the wine and juice industry, the textile industry, and also for improving the digestibility of vegetable sources, essentially in animal feed. Filamentous fungi produce enzymes used in a variety of these industrial processes, but also produce antibiotics, for example penicillin. Genetic transformation of filamentous fungi was generally performed in order to improve the production of penicillin or other high value compounds (EP 0 235951 and EP 0 260762). However, compared to penicillin or pharmaceutical proteins, which are high value compounds, enzymes used in industrial processes are of lower commercial value and their production has to combine high yields and low cost. In consequence, there is a need for a filamentous fungus able to industrially produce proteins of interest used in industrial processes, and particularly in animal feed and human food.

The filamentous fungus P. funiculosum is of great economical importance for its ability to produce a mixture of enzymes which can be used to increase the digestibility of animal feed. However, a better yield and control of enzyme production using P. funiculosum could be obtained by transforming P. funiculosum strains with specific transcription activating sequences functional in P. funiculosum to allow the expression of heterologous genes or to alter expression of homologous genes. To achieve this goal, a set of tools are needed. These tools include a method for transforming P. funiculosum, a selection marker for selecting transformants, and expression cassettes comprising regulatory elements functional in P. funiculosum.

Until now, no transformation method has been described so far for P. funiculosum. Furthermore, genetic transformation can be achieved either by complementation of an auxotrophic mutant towards prototrophy introducing the lacking anabolic gene (auxotrophic marker gene) or by genes conferring resistance against selection agents (dominant marker genes) to identify and isolate the transformed individuals. Auxotrophic complementation requires the possession of an auxotrophic strain carrying a mutation in a gene involved in a metabolic pathway and the corresponding gene which should complement the auxotrophic strain to prototrophy. This method was described in Yelton et al. (1984; Proc. Natl. Acad. Sci. USA; Vol. 31; 1470-1474) and Ballance and Turner (1985; Gene; 36; 321-331) for Aspergillus nidulans, and in Sanchez et al. (1987; Gene; 51; 97-102), Cantoral et al. (1987; Bio/Technology; 5; 497-497), EP 0 235951 and EP 0 260762 for the production of recombinant P. chrysogenum in order to increase penicillin production. Auxotrophic complementation has not yet been used to transform P. funiculosum.

An alternative to the use of auxotrophic complementation is the use of dominant selection markers. Numerous dominant selection markers are available and have been used to perform the transformation of several fungi, like hygromycin B phosphotransferase (Punt et al., 1987; Gene, 56(1):117-24), streptothricin acetyltransferase, phleomycin-resistance polypeptide and benomyl resistant beta-tubulin (Gold et al., 1994; Gene, 142(2):225-30), acetamidase (Beri and Turner, 1987; Curr Genet, 11(8):639-41), bialaphos-acetyltransferase (Avalos et al., 1989; Curr Genet, 16(5-6):369-72), but have not yet been used on P. funiculosum. The advantage of dominant selection markers is that they can instantly be used without previous selection for auxotrophic mutant strains.

Once transformation and selection methods are available, efficient regulatory elements are important to enhance and control the expression of nucleic acid sequences encoding homologous or heterologous proteins of interest. Efficient regulatory elements are promoter and terminator sequences allowing expression of the desired nucleic acid sequences of interest in a selected organism. Other genetic elements of interest are nucleic acid sequences encoding signal peptides that direct the secretion of the said protein of interest into the culture medium. To achieve this goal, such genetic elements need to be functional in the organism to be transformed, and may be isolated from it. Some genes have already been isolated from P. funiculosum, but no regulatory genetic tools have been obtained.

In consequence, the technical problem to be solved by the present invention is the preparation of a recombinant P. funiculosum in order to improve and control the industrial production of homologous or heterologous proteins.

The present invention relates to a recombinant P. funiculosum strain comprising at least one expression cassette functional in P. funiculosum stably integrated into its genome for the production of homologous or heterologous proteins.

The term “recombinant” applied to an organism means that this organism may contain at least one heterologous gene stably integrated into its genome, one extra copy of an homologous gene stably integrated into its genome, or one homologous gene whose regulation has been artificially modified. In the particular case of this invention, this means that the recombinant P. funiculosum contains at least one expression cassette stably integrated into its genome. To be stably integrated into the genome, the expression cassette is necessarily integrated by an artificial genetic mean using molecular and cellular biology techniques known to the one skilled in the art, artificial meaning different than nature-based methods such as hybridisation and selection. An expression cassette of the invention contains genetic elements, such as at least a promoter, a coding sequence and a terminator region operably linked in such a way that they are functional in P. funiculosum. Accordingly, the genetic elements constituting this expression cassette may each originate from P. funiculosum or may originate from other organisms provided that they are functional in P. funiculosum. Functional means allowing the expression of the coding sequence when the recombinant P. funiculosum is placed in conditions suitable for the expression of said expression cassette. For example, an expression cassette of the invention can comprise regulatory genetic elements, i.e. a promoter, a sequence encoding a signal peptide, and a terminator, originating from a P. funiculosum gene, operably linked to a coding sequence from another species, i.e. encoding a heterologous protein. Another expression cassette of the invention can comprise regulatory genetic elements originating from other species, provided that they are functional in P. funiculosum, operably linked to the coding sequence of a P. funiculosum gene, i.e. encoding a homologous protein, or to the coding sequence of a gene of another species, i.e. encoding a heterologous protein. The design and construction of such expression cassettes use standard molecular biology techniques known to the person skilled in the art (Sambrook et al., 1989, Molecular Cloning: A Labratory Manual).

In a preferred embodiment, the expression cassette contained in the recombinant P. funiculosum comprises, at least, operably linked in the direction of transcription, a promoter functional in P. funiculosum, a nucleic acid sequence encoding a homologous or heterologous protein and a terminator region functional in P. funiculosum. According to the present invention, a homologous protein is a P. funiculosum protein, and a heterologous protein is a protein originating from an organism different than P. funiculosum. Furthermore, the term “operably linked” means that the genetic elements are associated in a functional manner allowing the expression of the coding sequence.

In a preferred embodiment, the promoter is a promoter of a P. funiculosum gene. Preferably, the promoter comprises a nucleic acid sequence selected from the sequences disclosed in SEQ ID NO:1, SEQ ID NO:2, and SEQ ID NO:3, or a nucleic acid sequence hybridising to any one of these sequences.

According to the present invention, “hybridising” and “hybridisation” refer to nucleic acid sequences that are able to selectively hybridise with any one of the identified sequences (SEQ ID NO) at a level significantly higher than the background noise, and that are sharing the same function as said identified sequences. The background noise can be due to hybridisation of other nucleic acid sequences, in particular cDNA or genomic fragments present in cDNA or genomic libraries. The level of the signal generated by the interaction between the sequences able to selectively hybridise and the identified sequences (SEQ ID NO) of the invention is generally 10 times, preferably 100 times higher than that resulting from the interaction of the identified sequences with the sequences generating the background noise. The interaction level can be measured, for example, by labelling a hybridisation probe with the radio-element ³²P, the hybridisation probe being a sequence identified in the present invention or a fragment thereof. Selective hybridisation is generally obtained using stringent conditions (for example NaPO₄ 0,5M, 7% SDS at 60° C.-70° C. and pH 7,0). Hybridisation is realised using classical methods comprised in the state of the art (Sambrook et al., 1989, Molecular Cloning: A Labratory Manual).

In a preferred embodiment, the nucleic acid sequences hybridising to any one of the identified sequences are similar to said identified sequences. In the present invention, “similar” apply to nucleic acid sequences sharing one or more nucleotide modification compared to the identified sequences (SEQ ID NO). These modifications may be obtained by standard mutation methods, or chosen for the design of artificial oligonucleotide sequences used as probes in a hybridisation process or primers in polymerase chain reaction (PCR). The degree of similarity is expressed by the percentage of identical nucleotides between a similar sequence and an identified sequence. The methods for measuring and calculating sequence similarity are well known in the state of the art, and the man skilled in the art can, for example, use the PILEUP or BLAST programs described in Altschul & al. (1993, J. Mol. Evol. 36:290-300) and in Altschul & al. (1990, J. Mol. Biol. 215:403-10). Preferably, the degree of similarity between the similar sequences and the sequences identified in the present invention should be at least of 70%, most preferably of 80%, and even more preferably of 90%. Preferably, nucleotide modifications affecting similar sequences are neutral modifications, i.e. they do not affect the functionality of the sequences. For example, a nucleic acid sequence similar to a promoter of the invention keeps the same specific promoter function while sharing nucleotide differences with said promoter of the invention.

In a preferred embodiment, the terminator region is a terminator region of a P. funiculosum gene. Preferably, the terminator region comprises a nucleic acid sequence selected from the sequences disclosed in SEQ ID NO:4, SEQ ID NO:5, and SEQ ID NO:6, or a nucleic acid sequence hybridising to any one of these sequences.

The promoter disclosed in SEQ ID NO: 1 and the terminator region disclosed in SEQ ID NO: 4 derive from the Histone H4b gene. Expression cassettes constructed with this promoter permit a constitutive expression of homologous or heterologous proteins in recombinant P. funiculosum.

The promoter disclosed in SEQ ID NO: 2 and the terminator region disclosed in SEQ ID NO: 5 derive from the Acidic Aspartyl Protease gene. Expression cassettes constructed with this promoter permit a casein-regulated expression of homologous or heterologous proteins in recombinant P. funiculosum, said casein strongly increasing the expression.

The promoter disclosed in SEQ ID NO: 3 and the terminator region disclosed in SEQ ID NO: 6 derive from the csl31 gene. Expression cassettes constructed with this promoter permit a Corn Steep Liquor (CSL)-regulated expression of homologous or heterologous proteins in recombinant P. funiculosum, said CSL strongly increasing the expression.

In a most preferred embodiment, the recombinant P. funiculosum of the invention contains an expression cassette comprising a promoter and a terminator region of the same gene.

The recombinant P. funiculosum of the present invention contains an expression cassette comprising a nucleic acid sequence encoding any homologous protein. For example, the nucleic acid sequence encoding a homologous protein may encode a xylanase, a cellulase, a β-glucanase, or a ferulic acid esterase, or any cell-wall degrading enzyme isolated from P. funiculosum.

The recombinant P. funiculosum of the present invention may also contain an expression cassette comprising a nucleic acid sequence encoding any heterologous protein. The heterologous protein may be a fungal protein, a bacterial protein, a plant protein, an animal protein, or a protein from unidentified origin. For example, the nucleic acid sequence may encode a heterologous protein of interest such as a xylanase, a cellulase, a β-glucanase, a ferulic acid esterase, a pullulanase, an amidase, a phosphatase, a phytase, a mannanase, a milk-cloating enzyme isolated from any species. The nucleic acid sequence of interest may also have been obtained after screening of DNA libraries, encompassing cDNA or genomic DNA from various sources, including DNA libraries obtained by combinatorial or conventional mutagenesis of a given sequence or after directed molecular evolution (Arnold et Volkov, 1999, Current Opinion in Chemical Biology 3: 54-59; Skandalis et al., 1997, Chemistry & Biology 4: 8889-898; Crameri et al., 1998, Nature 391: 288-291) or after screening of libraries made with DNA from soil or other environmental samples. Without restriction to the following list, the nucleic acid sequence may encode a bacterial pullulanase like the one described in Yamashita et al. (1994, J Biochem, 116(6):1233-40), a bacterial amidase (Wyborn et al., 1996; Eur J Biochem, 240(2):314-22), a bacterial phosphatase or phytase (Kim et al., 1998, FEMS Microbiol. Letters 162: 185-191), a bacterial mannanase (Morris et al., 1995, Appl. Environ. Microbiol., 61: 2262-2269). It may also encode a xylanase, a cellulase, a β-glucanase, a ferulic acid esterase, a phytase (Pasamontes et al., 1997, BBA 1353: 217-223), a mannanase (Sachslehner et al., 1998, Appl. Biochem. Biotechnol. 70: 939-953) from fungus, a phytase (Maugenest et al., 1997; J Biochem, 322:511-17) or a chitinase (Boller et al., 1983; Planta, 157:22-31) from plant, a milk-cloating enzyme like chymosine from bovine (EP077109A2), or a reporter protein, for example the bacterial β-glucuronidase from Escherichia coli (Robert et al., 1989; Curr Genet, 15:177-80) or the green fluorescent protein as a marker for gene expression (Chalfie et al., 1994; Science, 263(5148):802-5).

According to the present invention, the recombinant P. funiculosum further contains an expression cassette comprising a signal sequence and a pro-sequence inserted between the promoter and the nucleic acid sequence encoding a homologous or heterologous protein. The signal sequence encodes a signal peptide which is directing the secretion of the expressed protein into the culture medium, and the pro-sequence encodes a pro-peptide which, when it is linked to an enzyme, maintains the enzyme in an inactive state until it is cut off by a protease. The association of such genetic elements to the sequences encoding the desired homologous and heterologous proteins is very useful to direct the secretion of the protein into the culture medium and control the timing of its biological activity. In a preferred embodiment, these genetic elements are from a P. funiculosum gene. Most preferably, they are isolated from the Acidic Aspartyl Protease gene and correspond to the nucleic acid sequence disclosed in SEQ ID NO: 7, or from the csl31 gene and correspond to the nucleic acid sequences disclosed in SEQ ID NO:8 or SEQ ID NO:9, or from nucleic acid sequences hybridising to SEQ ID NO:7, SEQ ID NO:8, SEQ ID NO:9.

The recombinant P. funiculosum of the invention may also comprise an expression cassette containing a nucleic acid sequence encoding a dominant selection marker as homologous or heterologous protein. Selection markers permit the selection of the transformed P. funiculosum, i.e. recombinant P. funiculosum, among those that are submitted to a genetic transformation process. In a preferred embodiment, the nucleic acid sequence encoding a dominant selection marker of the invention encodes an enzyme degrading a compound with an antibiotic or a fungicide activity. In a most preferred embodiment, the nucleic acid sequence encoding a dominant selection marker degrading a compound with antibiotic activity encodes an enzyme degrading hygromycin, kanamycin, oligomycin, streptothricin, or phleomycin. In a most preferred embodiment, the nucleic acid sequence encoding a dominant selection marker degrading a compound with fungicide activity encodes an enzyme degrading bialaphos (Avalos et al., 1989; Curr Genet, 16:369-72) or a beta-tubulin resistant against benomyl (Orbach et al., 1986; Mol Cell Biol, 6(7):2452-61).

The recombinant P. funiculosum of the invention may also comprise an expression cassette containing a nucleic acid sequence encoding a protein capable of complementing an auxotrophic P. funiculosum to prototrophy as homologous or heterologous protein. An auxotrophic P. funiculosum is a mutant carrying a mutation in a gene encoding an enzyme involved in a metabolic pathway. In order to restore prototrophy, such auxotrophic P. funiculosum needs to be complemented by genetic transformation with the functional gene corresponding to that carrying the mutation. Said functional gene may also share a mutation, but a mutation different from that shared by the auxotrophic P. funiculosum. In a preferred embodiment, the nucleic acid sequence encoding a protein capable of complementing an auxotrophic P. funiculosum to prototrophy encodes an enzyme implied in nucleoside biosynthesis or an enzyme implied in amino-acid biosynthesis. In a most preferred embodiment, the nucleic acid sequence is selected from pyrA, pyrB, pyrG, pyr4 (Buxton et Radford; 1983; Mol. Gen. Genet.; 190; 403-405), arg4, argB (Berse et al.; 1983; Gene; 25; 109-117), trpC (Goosen et al., 1989; Mol Gen Genet, 219:282-88) or the eukaryotic molybdopterin synthase (Appleyard et al., 1998; J Biol Chem, 273(24): 14869-76; Unkles et al., 1999; J Biol Chem, 274(27):19286-93) or mutated sequences thereof, provided that said mutated sequences share a mutation different from that shared by the auxotrophic mutant to be complemented.

The recombinant P. funiculosum of the invention may also comprise an expression cassette containing a nucleic acid sequence comprising the amdS gene (Corrick et al., 1987; Gene, 53:63-71; Medline: 87248110) allowing said recombinant P. funiculosum to grow on acetamide as a single N-source.

The present invention also relates to an expression cassette comprising, at least, operably linked in the direction of transcription, a promoter functional in P. funiculosum, a nucleic acid sequence encoding a homologous or heterologous protein and a terminator region functional in P. funiculosum.

In a preferred embodiment, the promoter and the terminator region are as described above.

In a most preferred embodiment, the expression cassette of the invention comprises a promoter and a terminator region of the same gene.

In a preferred embodiment, the expression cassette of the invention comprises a nucleic acid sequence encoding any homologous or heterologous protein as described above.

According to the present invention, the expression cassette further comprises a signal sequence and a pro-sequence inserted between the promoter and the nucleic acid sequence encoding a homologous or heterologous protein. Said signal sequence and a pro-sequence are as described above.

The expression cassette of the invention may also contain a nucleic acid sequence encoding a dominant selection marker, or a nucleic acid sequence encoding a protein capable of complementing an auxotrophic P. funiculosum to prototrophy as described above.

The present invention also relates to vectors containing an expression cassette according to the invention. Such vectors may be plasmids, phages or viruses, and they are used in the genetic transformation of host cells. Host cells containing these vectors are also encompassed by the present invention. These host cells may be fungal cells, bacterial cells, plant cells, or animal cells. A preferred fungal cell is a P. funiculosum cell.

The present invention also concerns a method for producing recombinant P. funiculosum. This method is a co-transformation method, that is a method leading to the uptake of at least one non-selected DNA, i.e. a plasmid or a DNA containing at least one expression cassette, and at least one other DNA fragment used for selection, i.e. plasmid or a DNA containing a gene encoding a dominant selection marker or a protein capable of complementing an auxotrophic P. funiculosum to prototrophy. The method also encompasses transformation which is essentially similar to co-transformation except that the DNA used for selection and the expression cassette are carried on the same piece of DNA.

First, this method consists in generating protoplasts of P. funiculosum. Generating protoplasts consists in the removing of the fungal cell wall by incubating the mycelia with specific enzymes using standard techniques well known to those skilled in the art.

Then, in a preferred embodiment, protoplasts are transformed in the presence of polyethyleneglycol (PEG) with at least two vectors containing an expression cassette according to the invention, one of these vectors containing a gene encoding a dominant selection marker. Alternatively, only one piece of DNA can be used provided it contains both an expression cassette according to the invention and a gene encoding a dominant selection marker. After transformation, recombinant P. funiculosum are selected with the selection agent corresponding to the dominant selection marker, and recovered recombinant P. funiculosum contain at least one expression cassette encoding the dominant selection marker and one expression cassette encoding a homologous or heterologous protein stably integrated into their genome.

In another preferred embodiment, protoplasts are transformed in the presence of polyethyleneglycol (PEG) with at least two vectors containing an expression cassette according to the invention, one of these vectors containing a gene encoding a protein capable piece of DNA can be used provided it contains both an expression cassette according to the invention and a gene encoding a protein capable of complementing an auxotrophic P. funiculosum to prototrophy. After transformation, recombinant P. funiculosum are selected with culture conditions corresponding to said prototrophy, and recovered recombinant P. funiculosum contain at least one expression cassette encoding the protein capable of complementing an auxotrophic P. funiculosum to prototrophy and one expression cassette encoding a homologous or heterologous protein stably integrated into their genome.

In another preferred embodiment, protoplasts are transformed in the presence of polyethyleneglycol (PEG) with at least two vectors containing an expression cassette according to the invention, one of these vectors containing the amdS gene. Alternatively, only one piece of DNA can be used provided it contains both an expression cassette according to the invention and a the amdS gene. After transformation, recombinant P. funiculosum are selected on culture medium containing acetamide as single N-source, and recovered recombinant P. funiculosum contain at least one expression cassette containing the amdS gene and one expression cassette encoding a homologous or heterologous protein stably integrated into their genome.

In P. funiculosum, co-transformation frequencies up to 95% are obtained. Preferably, co-transformations with a 1:1 ratio or a 1:3 ratio (selectable:non-selectable DNA) are performed.

Two other known transformation methods for producing recombinant fungi were tested to transform P. funiculosum.

The first method is described in Sanchez et al. (1998; Mol. Gen. Genet.; 258; 89-94) for the transformation of Aspergillus. It is called the restriction enzyme-mediated integration (REMI). This method consists in approximately the same method as the one described above which is the subject of the present invention, except that a mixture of restriction enzymes is added to the protoplasts, the DNA and the PEG during the transformation process. This method has led to an increased transformation frequency (20-60 fold) when applied to Aspergillus (Sanchez et al., 1998; Mol. Gen. Genet.; 258; 89-94). Applying the REMI transformation method to P. funiculosum, an increase in transformation frequency of 6-20 fold (6-20 transformants/μg pAN7-1) could be observed. However, using REMI, P. funiculosum transformants harbour one up to three copies of the gene in the genome. On the contrary, using the transformation method of the present invention, transformants containing up to more than 10 integrated copies of the gene in the genomic were obtained, allowing higher levels of production of a homologous or heterologous protein of interest. Therefore, the transformation method disclosed in the present invention confers an advantage compared to the REMI transformation method.

The second known transformation method is that disclosed in the European Patent n° EP 0260762. This method was applied to P. funiculosum but, after regeneration and selection, no candidate colonies were able to grow, i.e. were transformed, instead of nearly all with the method developed in the present invention. Accordingly, the transformation method disclosed in the European Patent n° EP 0260762 is not efficient in transforming P. funiculosum, while the transformation method disclosed in the present invention is particularly adapted to the transformation of P. funiculosum.

### EXAMPLES

### Example 1

### Culture and Protoplastation of P. Funiculosum

The fungus is cultivated on LYMM agar plates for 5 days at 25° C. During this time, sufficient amounts of conidia are produced. They were harvested in 0,01% Triton X-100 by pipetting up and down. 1 l Erlenmeyer flask with vexations containing 200 ml of MN-Uri broth (Punt, P. J. and van den Hondel, C. A. M. J. J. (1992) Methods in Enzymology 216, 447-457) was inoculated with 0,5-1,0 ml of the harvested conidia, to a final concentration of 10⁶ conidia/ml. Temperature of incubation was 28° C. for 24-28 hrs with an agitation of 220 rpm. LYMM contained (mg/ml): malt extract (10), yeast extract (1), Na₂HPO₄ (6), KH₂PO₄ (4), (NH4)₂SO₄ (2), MgSO₄.7H₂O (0.2), CaCl₂.7H₂O (0.001), H₃BO₃ (0.000001), MnSO₄.4H₂O (0.00001), ZnSO₄.7H₂O (0.00007), CuSO₄.5H₂O (0.00005), (NH4)₆MO₇O₂₄.4H₂O (0.0000123), FeSO₄.7H₂O (0.0001).

Germinated conidia were filtered through an autoclaved Nybold membrane (Ø 20 μm; SEFAR AG, Heiden, Swiss), then mycelium was rinsed, once with 20 ml water, once with 20 ml STC (1,13M Sorbitol; 10 mM Tris/HCl pH7,0; 50 mM CaCl₂). The mycelium was then incubated in 10 ml MgSO₄ Osmotikum (98 ml 1,2 M MgSO₄, 280 μl 1 M NaH₂PO₄, 1,670 ml 1 M Na₂HPO₄, 1 mlH₂O) completed with 10 mg,ml Novozym (Calbiochem) for 1,5 hrs at 28° C., 80 rpm in a sterile 30 ml pot. This mixture was then put in a 50 ml Falcon tube and carefully superposed with the same volume of Trap buffer (0,6M Sorbitol; 0,1M Tris/HCl pH7,0) before spinning at 1500 rpm, 10 min, in a Heraeus centrifuge. The brownish bottom phase contained the protoplasts. It was then transferred into a new 50 ml Falcon tube and gently mixed with 2× Vol STC before spinning at 2100 rpm, 10 min in a Heraeus centrifuge. The pellet was carefully resuspended in 10 ml STC, and the mixture spun at 1500 rpm for 10 min. If the yield should be increased, the centrifugation could be repeated with the supernatant at 2500 rpm. Finally, the pellets were resuspended in 0,5-1 ml STC. The final yield was 2,5×10⁸ protoplasts/ml.

### Example 2

### Transformation of P. funiculosum Protoplasts Using a Dominant Selection Marker

100 μl aliquots of protoplasts (10⁷-10⁸) were placed in 12 ml Falcon tubes and incubated with 5-10 μg DNA (pAN7-1; Punt et al., 1987; Gene, 56:117-24) for 20 min on ice. After this first incubation, 2 volumes of PEG (60% PEG 4000 (Merck); 0,1M Tris/HCl pH 7,0; 50 mM CaCl₂) were added, and the whole was gently mixed (pipetting up and down) and incubated for 10 min on ice. After incubation, 12 ml of hand-warm temperated Regeneration-agar (0,1% Yeast Extract; 0,1% Casein Hydrolysat; 34,2% Sucrose; 1,6% granulated agar) were poured onto the protoplasts, mixed by inversion 3-4 times and the mixture was immediately poured onto Petri dishes. The protoplasts were allowed to regenerate for 15-18 hrs at 25° C. without selection pressure. In the case of Hygromycin as selection marker, a concentration of 300 μg/ml for an overlay (12 ml) with 1% granulated agar revealed to be best. It was then further incubated at 25° C. for 2 days. A second overlay might be poured onto the plates (12 ml 1% granulated agar containing 500 μg/ml Hygromycin B). This step allows to focus the colonies of the transformants after further incubation colonies arose (1 transformant/μg DNA).

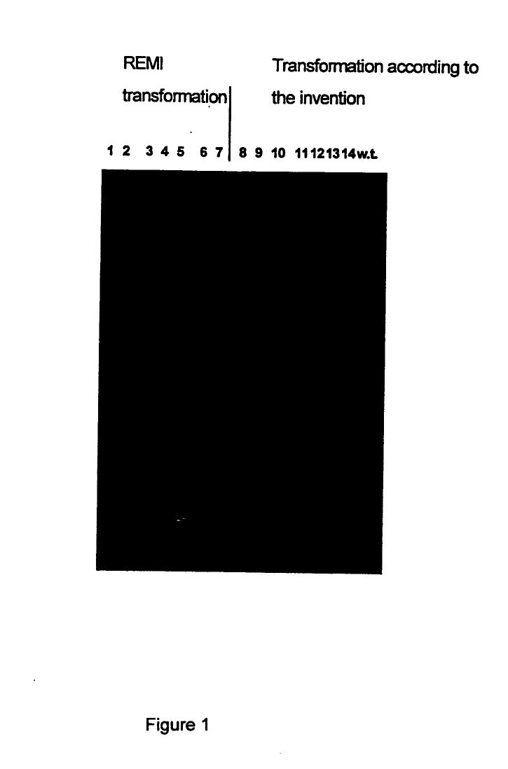

DNA of colonies appearing after 5 days on hygromycin B containing agar plates was extracted using standard molecular biology techniques (Sambrook et al., 1989, Molecular Cloning: A Labratory Manual). The DNA was digested with EcoRI, transferred onto a nylon membrane and hybridised with an EcoRI/BamHI DNA fragment of pAN7-1 containing the gene hph. After exposition, 14 out of 14 hygromycin B resistant colonies showed at least one integration of the plasmid pAN7-1 on the autoradiograph (FIG. 1).

### Example 3

### Construction of a P. Funiculosum Zenomic Library in XZAPII

100 ml of LYMM containing glucose (10 g/l) instead of malt extract (CM) was inoculated with 10⁸ conidia and incubated with rotation (150 rpm) at 30° C. for 3 days. The pH was adjusted to 5,0. Genomic DNA was isolated from the mycelia according to Raeder, V. and Broda, P. (1985; Lett Appl Microbiol, 1:17-20), re-suspended in 50 mM MOPS pH 7.0, 0.75M NaCl and applied to a plasmid purification column (QIAgen). The column was washed and the DNA eluted according to the manufacturer's instructions. The purified DNA was partially digested with EcoRI (2 U/μg DNA) at 37° C. for 25 minutes, extracted with phenol:chloroform (1:1, v/v) and electrophoresed using a 1% TAE-agarose gel (Sambrook et al., 1989, Molecular Cloning: A Labratory Manual). DNA fragments of length 3.5 to 12 kb were excised from the gel and purified using a QIAex gel extraction kit (Qiagen). The purified fragments were ligated to EcoRI/λZAPII vector arms (Stratagene), packaged into the phage and amplified according to the manufacturer's instructions. The final titre was determined at 1.2×10⁸ pfu/ml.

### Example 4

### Identification of the Histone H4b Gene and Isolation of its Promoter and Terminator Region

The P. funiculosum genomic library was screened for the histone H4 gene by PCR using the degenerate primers NB063 (SEQ ID NO:10) and NB064 (SEQ ID NO:11). Positive clones bearing the 7.6 kbp EcoRI insert were sub-cloned and the 2.3 kbp insert was sequenced. PCR amplification of the H4b promoter region (SEQ ID NO:1) was performed in a 50 μl volume containing 50 ng of template DNA, 250 nM of primers NB 156 (SEQ ID NO:12)/NB157(SEQ ID NO: 13), 200 μM dNTPs, 5 mM MgCl₂ and reaction buffer supplied by the manufacturer. After 3 min de-naturation at 94° C., 2U of DNA polymerase was added, followed by 30 cycles of 1 min at 94° C., 30s at 50° C., 1 min at 72° C. One tenth of each PCR reaction was analysed by agarose gel electrophoresis. The 450 bp fragment was gel-purified (QIAquick Kit, Qiagen Ltd., Dorking, U.K.), and SpehlEcoRI digested following the enzyme manufacturer's instructions (Promega, Madison, USA). The DNA fragment was once again column purified (QIAquick), ligated into pBluescript SK vector (Stratagene) (molar ratio vector to insert 1:5) and transformed into E. coli XL1-Blue (recA1 endA1 gyrA96 thi-1 hsdR17 supE44 relA1 lac [F′ proAB lacI^{q} ZΔM15 Tn10 (Tet^{r})]^{c} (Stratagene) using standard CaCl₂ protocol (Sambrook et al., 1989, Molecular Cloning: A Labratory Manual). Recombinant bacteria were grown on LB agar plates containing 100 μg/ml ampicillin overnight at 37° C. Screening was performed by PCR on isolated individual transformed colonies using the primers and conditions described above. The positive clones were grown in 100 ml LB broth (100 μg/ml ampicillin) and the plasmid DNA purified using Qiagen 100 columns according to the manufacturer's instructions (QIAgen). Sequencing was performed on an automated DNA sequencer using the Big Dye terminator cycle sequencing kit (Applied Biosystems).

PCR amplification and cloning of the H4b terminator region (SEQ ID NO:4) was performed as described above using primers MJA004 (SEQ ID NO:14)/MJA005(SEQ ID NO:15) and the restriction enzymes XhoI and KpnI.

### Example 5

### Identification of the Acidic Aspartyl Protease Gene and Cloning of a Genomic DNA Fragment Carrying the Promoter, Gene and 3′Terminal Region

PCR amplification of an internal fragment of apf was performed using the primer combination: PEP5′ (SEQ ID NO:16) and PEP3′ (SEQ ID NO:17). These degenerate primers were designed on the basis of those disclosed in Gente et al. (1997; Mol Gen Genet, 256:557-65) taking into account codon usage to lower the degree of degeneracy.

50 ng of genomic DNA from P. funiculosum IMI 134756 served as template. Further, we used 1 pmol of each primer, 2001M dNTPs in a Volume of 50 μl, superposed with mineral oil (Perkin Elmer). 5U Taq Polymerase (Appligen) were added after a denaturation step of 3 min at 94° C. The running conditions were 94° C. 1 min; 60° C. 1 min; and 72° C. 1 min for 35 cycles. The PCR sample was applied onto a 1% Agarose gel and only one DNA fragment of 620 bp in length appeared. It was cut out and eluted by the Oiaex II gel extraction kit (Qiagen). The purified DNA fragment was then ligated into the T-vector pGEM-Teasy (Promega). The resulting plasmid was named pPEP. Sequecing of the cloned fragment has been performed by Genome Express. Translation of the identified DNA sequence revealed strong homology to the Aspartyl Protease of P. roqueforti.

In order to check if apf is a single copy gene, the 620 bp DNA fragment of pPEP was isolated by an EcoRI digest, purified over gel and labelled with ³²p a-dCTP using the Megaprime labelling system (Amersham). Genomic DNA of IMI 134756 has been isolated and digested with the enzymes BglII, PstI, BglII/PstI, MluI, NcoI, SacII, NcoI/SacII and applied onto a 1% agarose gel. The gel was blotted onto a Hybond N Nylon membrane (Amersham) following the described protocols in Sambrook et al. (1989, Molecular Cloning: A Labratory Manual). Hybridisation using the labelled 620 bp EcoRI fragment as probe has been carried out over night at 65° C. in 0,5M NaPO₄, 7% SDS; pH7,0 Hybridisation buffer. Washing was performed twice at 65° C. for 1 min with 0 μM NaPO₄, 1% SDS; pH 7,0 Washing buffer. The film (Hyperfilm MP; Amersham) was exposed for 3 hrs. After development only one single signal was clearly visible in each lane, indicating that apf is indeed a single copy gene.

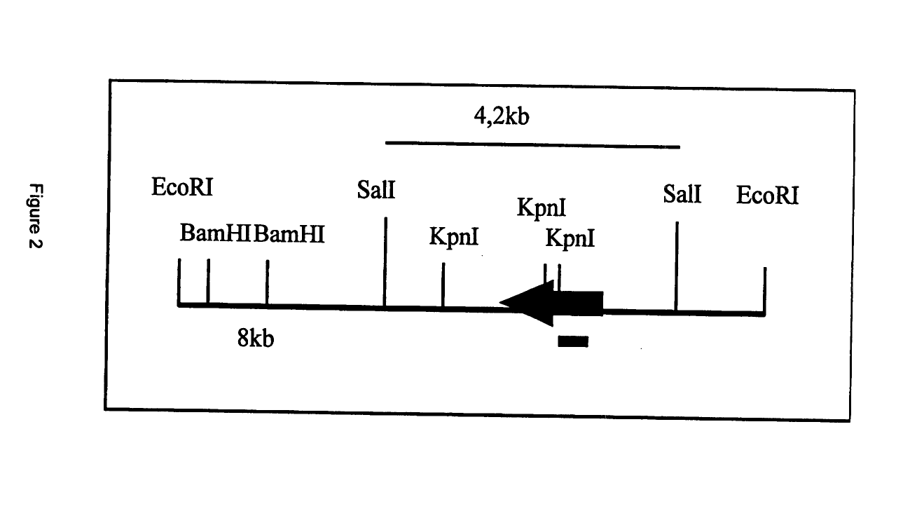

The screen of our genomic lambda ZAPII library using a 450 bp EcoRI/KpnI fragment of the 620 bp PCR apf product as probe comprehended approximately 90 000 phages which led to the identification of 15-20 phages carrying a DNA hybridising with the apf probe. The isolated plasmid has a size of 11 kb, carrying a genomic EcoRI insert of 8 kb. Several restriction enzymes have been used to draw a restriction map of this EcoRI fragment (FIG. 2). We have sequenced the genomic region of the apf gene including the promoter (SEQ ID NO: 2), the sequence encoding the signal peptide and the pro-peptide (SEQ ID NO: 7), and the terminator region (SEQ ID NO: 5).

### Example 6

### Identification of the csl31 Gene

Identification of the Strongly Secreted 31 kDa Protein in Corn Steep Liquor (CSL) Media

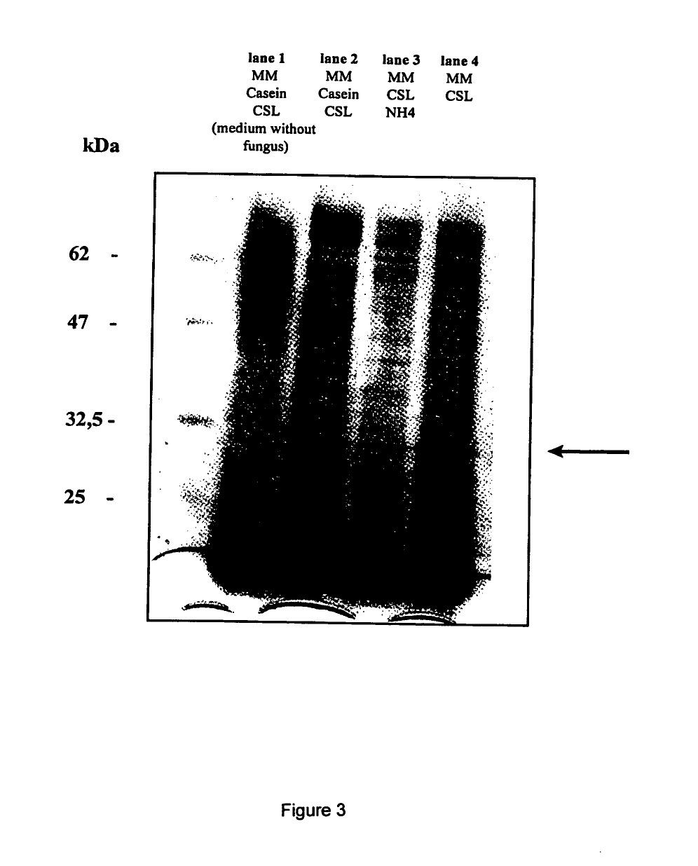

Proteins of the supernatant separated on a SDS-PAGE deriving from a two days old culture of minimal medium, containing only CSL and Casein revealed the presence of a prominent protein of 31 kDa. In order to verify if this protein is a degradation product from Casein, proteins of a set of different minimal media were analyzed two days after inoculation. As a control minimal medium containing Casein and CSL incubated without P. funiculosum was used (FIG. 3, lane 1). Finally, even in minimal medium containing only CSL, this protein could be observed (FIG. 3, lane 4). This indicates that CSL as substrate induces the production of this fungal protein. A slight reduction of the protein could be noted in a minimal medium containing CSL and Ammonium (FIG. 3, lane 3).

Protein sequencing of the N-terminus had been initiated. An aliquot of filtrated supernatant form cultures incubated for 48 hrs at 28° C., 190 rpm was applied onto a 10% SDS-PAGE. The proteins were transferred from the gel onto a PVDF membrane using the semi-dry ProBlott system (Applied Biosystems). The membrane was Amido black stained (45% Methanol; 1% acetic acid; 0,1% amido black) and washed in water. After drying, the stained protein band was cut out of the membrane and sent for micro-sequencing. Protein micro-sequencing was performed by the Institute Pasteur (Paris; see analyze 98C1148 as reference). The resulting sequence was: XXYQTRIFEAGTTFG (SEQ ID NO:18).

On the basis of the amino acid sequence of the 31 kDa protein (which is named Csl31 for Corn Steep Liquor induced 31 kDa protein) identified by N-terminal sequencing, a 3′RACE (rapid amplification of cDNA ends) was performed to reveal the csl31 gene. The principle of the 3′RACE is the use of a polydT anchor primer (QT: SEQ ID NO:19), following a reverse transcription of mRNA. In a first round of amplification, the outer part of this QT primer represent an annealing site for the primer QO (SEQ ID NO:20). The corresponding primer in the N-terminal of Csl31 covers the sequence QTRIF (GSP12: SEQ ID NO:21). An aliquot of this PCR is then used as template in a second amplification by the primer Qi (SEQ ID NO: 22=inner part of the QT anchor sequence) and the primer GSP22 (SEQ ID NO:23=corresponding the sequence EAGTT of Csl31).

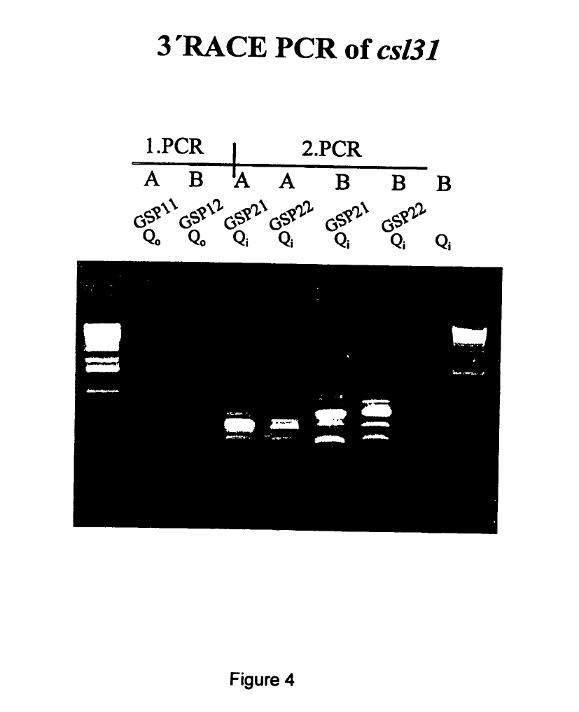

In this way, two DNA fragments of 750 bp have been amplified in the primer combination Qi/GSP21 and Qi/GSP22 (FIG. 4). They have been eluted and ligated into the vector pGEM T-easy (Stratagene) and sequenced. A translation of this sequence reveals the presence of an ORF, where the sequence of the primer GSP22 can be found. Surprisingly, according to the N-terminal sequence of Csl31 the predicted glycine after the phenylalanine is not present. Furthermore, the length of the coding sequence (480 bp) does not correspond to the predicted size of Csl31 (250aa, that means 750 bp).

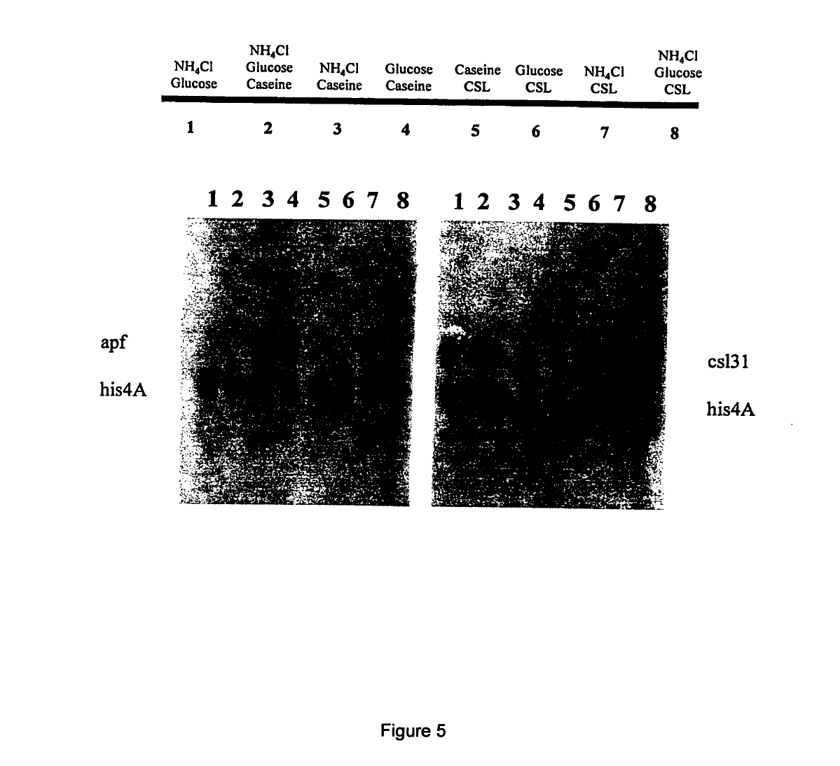

When the cloned 3′RACE fragment is used as probe on a Northern blot, the estimated size of the csl31 mRNA is about 900 bp (FIG. 5).

Identification of the Genomic Sequence of csl31

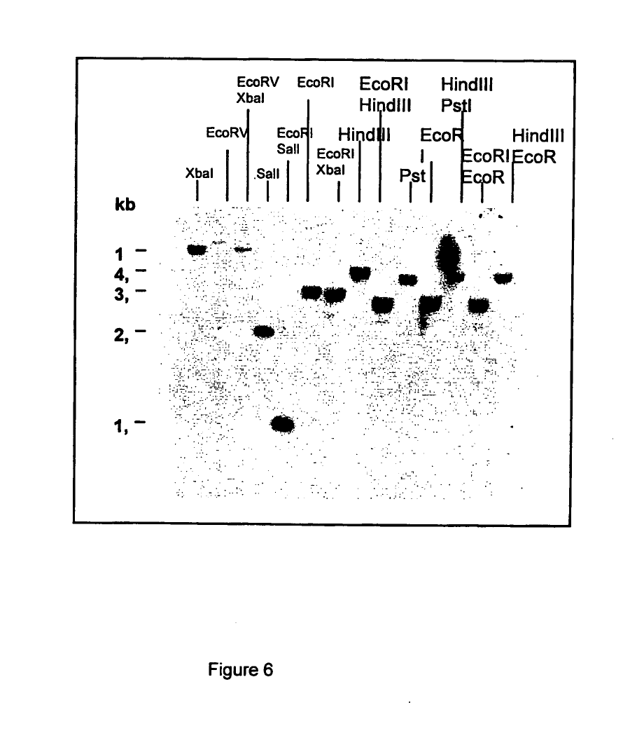

Southern analysis has revealed that the gene csl31 is located on a 3,4 kb EcoRI fragment (FIG. 6). Since our AZAPII library is size limited (see example 3), we decided to construct a genomic cosmid library of P. funiculosum IM1134756 in order to identify the csl31 gene together with its promoter and terminator.

Genomic DNA of the strain P. funiculosum IMI134756 has been partially digested with 0,8U SalI for 20 and 40 minutes and with 2,8U for 40 minutes at 37° C. Fragments had been separated on a CHEF gel for 20 hours (5V/cm; switch time of 8sec and a migration angle of 120°). DNA of the size between 25 and 60 kb has been cut out, B-Agarase digested and ligated into the XhoI site of the vector pMOCosX (M. Orbach; Gene (1994), 150, 159-162). The Gigapack Gold packaging protocol from Stratagene has been followed to encapsidate the cosmids and transform them into the strain Ecoli Q358 (hadR, supE, +80r; Ref.: Maniatis et al., 1982).

Size of the library: 50×95 single E. coli colonies were picked and transferred into microtiter wells. Around 2000 remaining colonies were pooled by rinsing the LB agar plates. They are stored in 10 glycerol stocks. An analysis of eight randomly picked clones revealed different restriction pattern and an average insert size between 40 and 45 kb. Taking the calculations of Seed et al. (1982; Gene, 19, 201-209) into account, our library will cover the genome of P. funiculosum with a probability of more than 95%.

Identification of the Cosmid 18B5, Containing the csl31 Gene

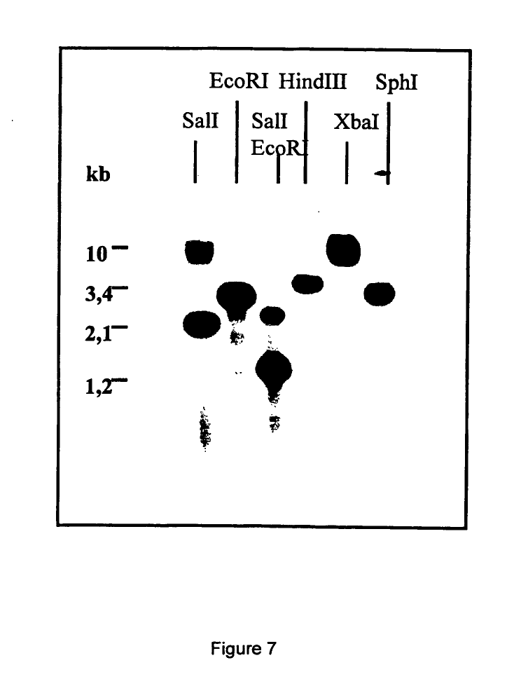

A 600 bp EcoRI/BglII fragment (deriving from the RACE screen) containing the 3′ end of the csl31 gene was used as probe to screen the cosmid library. The pool filters and the individual filters from each microtiter plate were hybridized first, hybridizing the first 20 filters at the same time with the csl31 RACE probe. One clear signal could be obtained for filter no18. DNA of the respective E. coli clone (B5) was isolated, digested and analyzed on Southern blot (FIG. 7). The obtained signal pattern was identical to that previously observed on the genomic Southern blot. Following the established restriction map, a 3,6 kb SphI fragment carrying the csl31 gene in the middle was cloned.

Sequence of the csl31 Genomic Fragment

The 3,6 kb SphI genomic subclone of csl31 was cloned and entirely sequenced. This fragment consists of 1350 bp promoter region (SEQ ID NO: 3), 912 bp csl31 open reading frame and 1350 bp terminal region (SEQ ID NO:6).

Sequence analysis of the csl31 gene revealed an open reading frame coding for a protein of 303aa. There were no indications for the presence of intron sequences (neither 5, nor 3, splice sites). Finally, the N-terminal sequence of the Csl31 protein was identified. In addition, the entire sequence of the cDNA RACE fragment was found. This has proved without doubt the fact that only half of the csl31 gene has been cloned in the RACE experiment.

In the complete sequence two more repeats appeared. The Csl31 protein displays really a highly modular structure. Therefore it is quite comprehensible that the degenerated N-terminal primers have found multiple annealing sites in the cDNA template. A favored amplification of one of these five N-terminal regions was the consequence.

### Example 7

### Construction of Histone Expression Cassettes

Construction of Xylanase+Histone Promoter and Terminator Plasmid

H4b promoter plasmid pMJA001 was constructed by assembling the amplified PCR fragment containing the H4b promoter region into Bluescript SK plasmid (Stratagene Ltd, Cambridge, U.K.).

H4b promoter+H4b terminator plasmid pMJA007 was constructed by assembling the amplified PCR fragment containing the H4b terminator region into pMJA001 plasmid. A MCS (Multiple cloning site) containing the following restriction sequences: EcoRV, Hind III, Cla I, Bsp106 I, Sal I, Acc I, Hinc II was inserted between the promoter and terminator sequences of H4b.

The plasmid pMJA003 containing the Xylanase ORF and the histone H4b promoter and terminator was constructed by assembling the PCR amplified fragment containing the Xylanase ORF into the pMJA007.

The P. funiculosum phage genomic library was screened with the redundant primers Furniss 3 (SEQ ID NO:24) and 5 (SEQ ID NO:25). The 2.9 kbp Xylanase C sub-fragment was contained in a 8 kbp EcoRI insert. Essentially, PCR amplification and cloning of the Xylanase C ORF region was performed using primers MJA001 (SEQ ID NO:26)/MJA003 (SEQ ID NO: 28) and the restriction enzymes EcoRI and XhoI.

Construction of Histone promoter+Xylanase C Gene Plasmid

The plasmid pMJA008 containing the Histone H4b promoter, Xylanase ORF and Xylanase terminator was constructed by assembling the PCR amplified fragment containing the Xylanase ORF and Xylanase terminator into pMJA001.

Essentially, PCR amplification and cloning of the Xylanase C gene was using primers MJA001(SEQ ID NO:26)/MJA002(SEQ ID NO:27) and the restriction enzymes EcoRI and XhoI.

Construction of Histone H4b Promoter+uidA ORF+Histone Terminator Plasmid

The construction of the histone driven uidA reporter gene plasmids started with the construction of the H4b-vector.

PCRs were performed with pBS-H₄B (the original library clone containing the H₄B gene) as template and NB116(SEQ ID NO:29)/NB117(SEQ ID NO:30) for the H₄B promoter or NB118(SEQ ID NO:31)/T7 sequencing primer for the H₄B terminator. The PCR products were gel electrophoresed and DNA fragments corresponding to predicted sizes purified and ligated to pGEM-T-EASY. The resultant plasmids (pNJB61 containing H4b promoter and pNJB62 containing H4b terminator) were checked by restriction digests.

pNJB62 was digested with BamHI and the resultant 2.9 kbp fragment containing H4b terminator was gel purified and ligated to BamHI cut/phosphatased pNJB61 to give pNJB63. The unwanted BamHI site in pNJB63 was removed by digesting with XbaI/PstI, blunt-ending with T4 DNA polymerase and re-ligating to give pNJB64.

On the basis of pNJB64, a H4b promoter+uidA+H4b terminator vector was constructed. Plasmid pAN52-7 carrying the E. coli uidA gene on a NcoI fragment was gel purified and the uidA fragment blunted by Mung bean digestion. The product was then ligated to BamHI cut/Mung bean blunt ended pNJB64 to give pNJB68. The success of all ligations was checked by sequencing.

In order to remove the H3 gene region, present at 5′ of the H4b promoter, pNJB68 was digested with SpeI and NcoI and after gel purification, re-ligated originating the pNJB69.

### Example 8

### Construction of Aspartyl Protease Expression Cassette (PBAG)

The construction of the expression cassette pBAG was initiated by PCR, using the plasmid papf (8 kb EcoRI) as template. This plasmid contains the whole 8 kb apf genomic sequence including 2,2 kb promoter region.

The promoter region was amplified using the primer M13(−20) as standard universal primer and ApfA (SEQ ID NO:32) cutting off the putative pre-pro sequence of Apf at position aa 68. The altered aa sequence is therefore SAASM instead of SAAAS. PCR conditions were 1× Taq Pol. Buffer;1 pmol of each primer; 200 μM dNTPs; 5U Taq Polymerase (Appligen) in 50F1 Volume. The resulting 2,4 kb PCR fragment was ligated into pGEM T-easy. The plasmid containing this insert was named pP1.

The terminator region of apf was amplified by PCR using the vector pBIISK-containing a 4,2 kb SaII genomic subclone of apf. As primer, again the primer M13(−20) could be used and the primer ApfB (SEQ ID NO:33) carrying the Stop codon (TAA) of apf The PCR was performed as described above. The 2,2 kb PCR DNA fragment was ligated into the vector pGEM T-easy and the resulting plasmid named pT7.

As reporter gene, the uid4 gene deriving from the vector pNOM102 (Roberts et al., 1989; Curr. Genet. 15, 177-180) was used as NcoI fragment. As vector, pUC19 was digested by EcoRI/SaII. The plasmid pP1 carrying the promoter region was restricted by NcoI/EcoRI and the fragment was purified. The plasmid pT7 containing the terminator region of apf was digested with SaII/NcoI and this 2,2 kb DNA fragment was also purified.

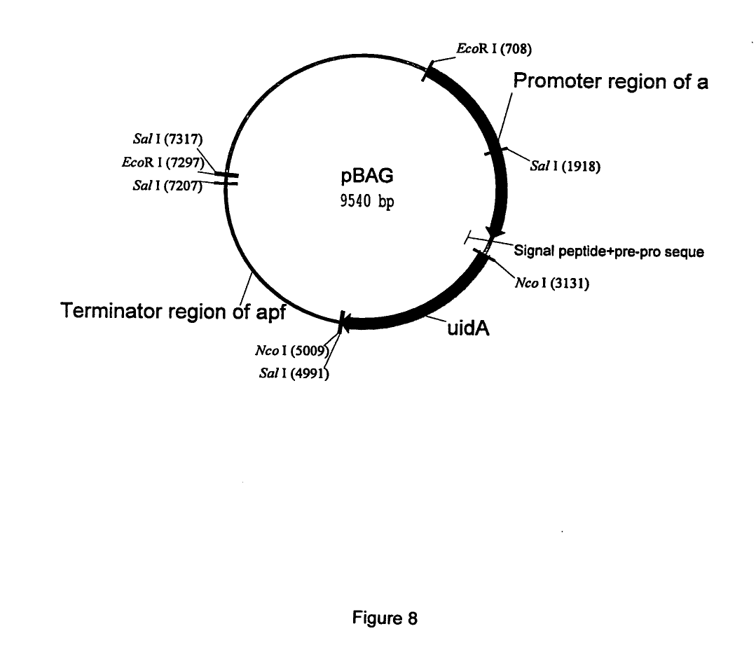

Ligation of those four DNA fragments was performed, resulting in the plasmid pBAG. The plasmid is shown in FIG. 8.

### Example 9

### Construction of csl31 Expression Cassette (pBCGT)

The identified 3,6 kb SphI fragment ligated into pUC19 encompassing the csl31 gene served as template for the construction of the csl31 expression cassette. Two primer combinations were used to amplify the promoter and terminator region of csl31. The oligonucleotids CslDel1 (starting at the identified N-terminal region of the gene transcribing in upward direction) and CslDel2 (starting at the Stop codon of Csl31 transcribing downstream) are both carrying a NaeI site which is thought to be used to cut and religate promoter and terminator region of csl31, thereby deleting the csl31 gene. The promoter region was amplified by PCR [94°, 1 min; 55°, 1 min; 72°, 2 min; 30 cycles] with the primer combination CslDel1(SEQ ID NO:34)/M13reverse. The 1,5 kb DNA fragment was purified over gel and cloned into pGEM T-easy (Promega). One clone has been chosen, sequenced and named pP3.

The terminator region was also amplified by PCR [94°, 1 min; 55°, 1 min; 72°, 2 min; 30 cycles] with the primer combination CslDel2 (SEQ ID NO:35)/M13(−20). The resulting 1,3 kb DNA fragment was purified over gel and cloned into pGEM T-easy (Promega). The plasmid has been named pT2 and sequenced.

The csl31 cassette was constructed by ligating the SphII-SpeI 1450 bp promoter fragment of pP3 together with the SpeI-PvuII 1250 bp terminator fragment of pT2 into SphI-SmaI pUC19. The resulting plasmid was named pUCE2. Sequencing revealed that only the NaeI site of CslDel1 remained in this construct.

Because of the uncompleted digestion of pUCE2 by Nael, we changed the NaeI restriction site into a SmaI site. This was carried out by PCR [94°, 1 min; 50°, 1 min; 72°, 3 min; 30 cycles], amplifying the promoter region of pUCE2 by the primer combination CSL12 (SEQ ID NO:36)/CSL13 (SEQ ID NO:37). The 1 kb NcoI/SpeI fragment of pUCE2 was exchanged by the NcoI/SpeI digested PCR product. The resulting plasmid was named pUCE1. Direct insertion of the uidA reporter gene into the SmaI site of pUCE1 was not possible. In order to do so, we reconstruct this expression cassette.

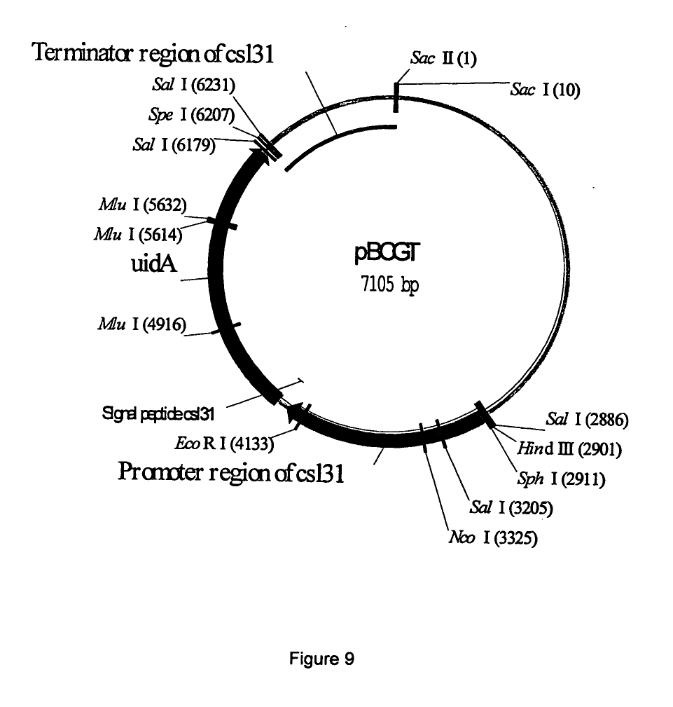

The HindIII/SmaI fragment of pUCE1 carrying the promoter and leader sequence of csl31, was ligated into HindIII/SmaI pBIISK-(Stratagene) resulting in the plasmid pBC3. The uidA reporter gene was cut out from pNOM102 by NcoI, blunt ended by Mung Bean nuclease and inserted into the SmaI site of pBC3 resulting in the plasmid pBCG8. The csl31 terminator region from pUCE1 was then isolated as SpeI/SacII fragment and ligated into SpeI/SacII restricted pBCG8. The resulting plasmid containing the csl31 expression cassette was named pBCGT (see FIG. 9).

### Example 10

### Co-transformation of the PBAG (Aspartyl Protease) Expression Cassette

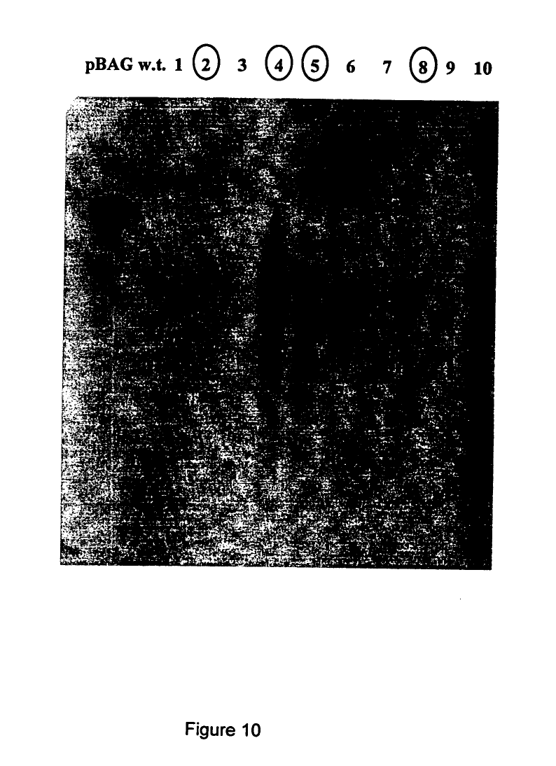

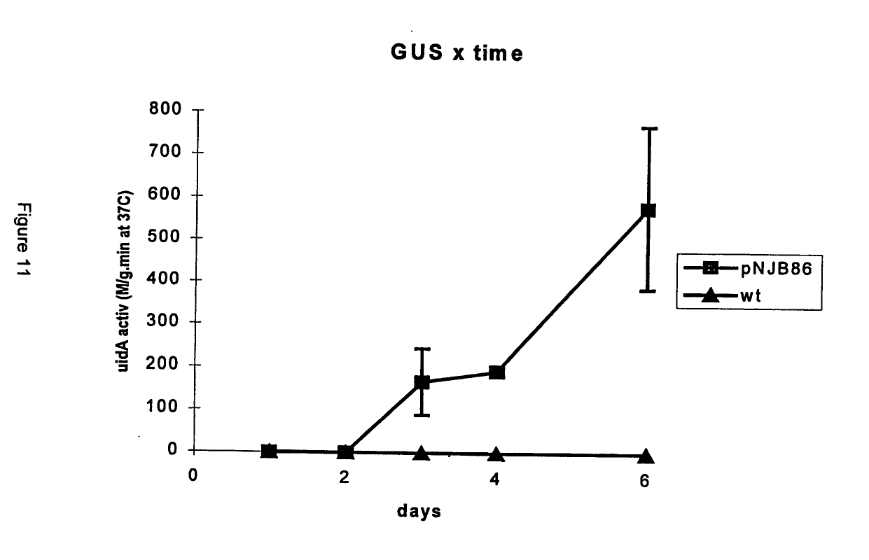

The plasmid pBAG was co-transformed with the plasmid pAN7-1 into P. funiculosum IMI134756. Hygromycin resistant colonies have been transferred onto LYMM agar plates containing 200 μg/ml Hygromycin B and analysed on Southern blot using the uidA gene as probe. 11 out of 12 hygromycin-resistant colonies carried the integrated plasmid pBAG. Four transformants (no2, no4, no5 and no8 see FIG. 10) have been further investigated towards their β-glucuronidase activity.

10⁵ conidia/ml of those four co-transformant strains were used to inoculate 50 ml MM-Casein medium (MM: 0,15% KH₂PO₄, 0,05% KCl, 0,05% MgSO₄, 0,001% MnCl₂, 0,001% FeSO₄, 0,001% ZnSO₄; 100 mM NH4Cl; 1% Casein) in 125 ml Erlenmeyer flasks. After 72 hrs at 28° C., 180 rpm the cultures were harvested by filtration through a 3MM Whatman paper. The filtrate was directly used for the enzyme assay. The obtained mycelium was resuspended in 1 ml Extraction buffer (50 mM sodium phosphate buffer pH7,0; 1 mM EDTA; 5 mM β-Mercaptoethanol; 0,005% Triton X-100), and sonicated (5 min, 50% active cycle, step2). After centrifugation, the clear supernatant was divided in two aliquots (one for β-glucuronidase determination, the second for a Bradford assay). A Bradford assay has been performed to determine the amount of protein according to the protocol of the manufacturer (Bio-Rad). Due to the presence of oligo-peptides in the filtrate (deriving from the hydrolyses of the Casein) a proper quantification of secreted fungal proteins was not possible. The activity assay for the B-glucuronidase has been carried out as described.

As result, we have obtained a very high B-glucuronidase activity in the medium and less in the analysed mycelium (see table 1).

TABLE 1
β-glucuronidase activity of pBAG co-transformed strains
in MM-Casein medium

| | Mycelium |
|---|---|
| Co-transformed strains | β-glucuronidase activity in |
| containing pBAG/pAN7-1 | μM/mg total prot./min |
| No 2 (1 integration) | 35 |
| No 4 (8 integration) | 600 |
| No 5 (8 integration) | 579 |
| No 8 (2 integration) | 99 |
| IMI (wild-type) | 0 |

In conclusion, the predicted leader and pre-pro sequence of the acidic Aspartyl Protease are functional and direct the secretion of the β-glucuronidase into the medium. Furthermore, the β-glucuronidase activity measured in the mycelium is almost proportional with the integration number of the plasmid pBAG. Therefore, the values reflect rather the strength of the Aspartyl protease promoter than the appearance of position effects.

### Example 1

### Co-Transformation of Histone Expression Cassettes

Co-transformation of pNJB68 (Histone) Expression Cassette

Four uidA transformants, obtained by co-transformation of pNJB68 and pAN7-1, were grown in minimal medium (1% glucose, 0.1M NH₄Cl) at 25° C. and the time course for the intracellular expression of uidA driven by the histone 4b promoter measured essentially as described by Roberts et al., 1989. Curr Genet. 15: 177-180 (FIG. 1).

Co-transformation of pNJB69 (Histone) Expression Cassette

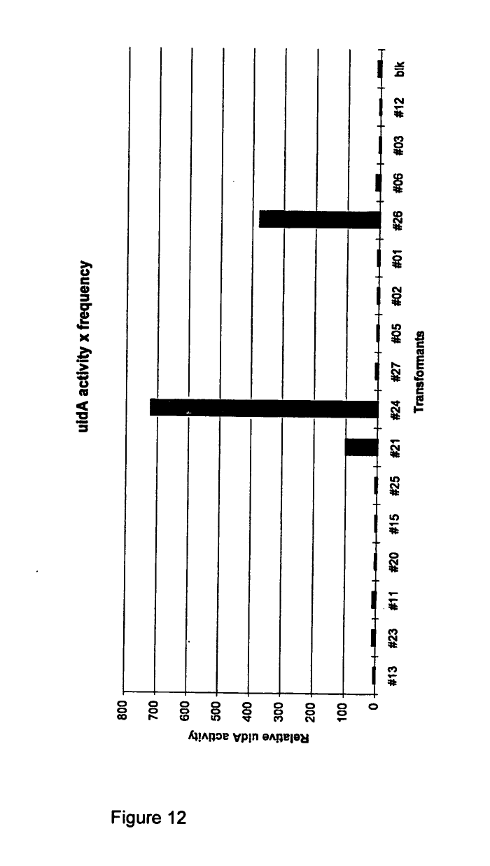

In order to remove the H3 gene region, present at 5′ of the H4b promoter, pNJB68 was digested with SpeI and NcoI and after gel purification, re-ligated originating the pNJB68. When spores of pNJB69 co-transformants were qualitatively evaluate for intracellular uidA activity (minimal medium after 5 days at 25° C.), 4 out of 16 transformants driven by histone 4b promoter have shown medium to high levels of activity (FIG. 12).

Co-Transformation of pMJA003 and pMJA008 (Histone) Expression Cassette

The constructs pMJA003 and pMJA008 was co-transformed with a pAN7-1 hygromycin vector into P. funiculosum. After selection on ‘Regeneration agar’ plates containing up to 300 μg/ml of Hygromycin B, putative transformants were allowed to sporulate on LYMM (300 μg/ml of Hygromycin B) slopes at 30° C. Individual spores were then isolated, grown in LYMM slopes and, strain specific spores stored at −70° C.

The transformants were screened for Xylanase activity by a modified version of the Congo Red polysaccharide interaction method, routinely used for lambda library screening at The Babraham Institute and originally described by R. M. Teather & P. J. Wood (1982) Applied and Environmental Microbiology, 43(4):777-780.

Essentially, soluble xylan is prepared by solubilising oat spelt xylan (10 g-Sigma X0627) in distilled water (250 ml), the pH adjusted to 10 with sodium acetate, and stirred at room temperature for 1 hour. The supernatant is collected by centrifugation (10,000 g for 10 min), neutralised to pH 7.0 with 1 M acetic acid and freeze dried.

The xylan overlay is then prepared by heating a solution of soluble xylan (0.25%) and agar (1%) in phosphate/citrate buffer (50 mM-pH 6.5). After cooling to c.a. 50° C., the xylan overlay is poured (4 ml) on top of MN-hygromycin B plates previously inoculated with P. funiculosum transformants and incubated at 30° C. for 2 days.

Staining of total polysaccharides is achieved by adding enough Congo red (1% in water) to cover the plate and incubating at room temperature for 15 min. The yellow halo of glucanohydrolase activity is then detected by pouring off the Congo red stain and repetitively de-staining with NaCl (292g/L)-Ammonia (0.880-2.5 ml/L).

Using official reducing group assay (DNS), six transformants have shown stable integrations and glucanohydrolase activities after 3 passages on MN-hygromycin B plates. The positive clones and a negative control were then grown in MN liquid media (50 ml-25° C./5 days) and the supernantant tested for Xylanase activity by the official DNS (Dinitrosalicylic acid) method (Baileys et al. (1992) Inter-laboratory testing of methods for assay of xylanase activity, J. Biotechnology 23:257-270).

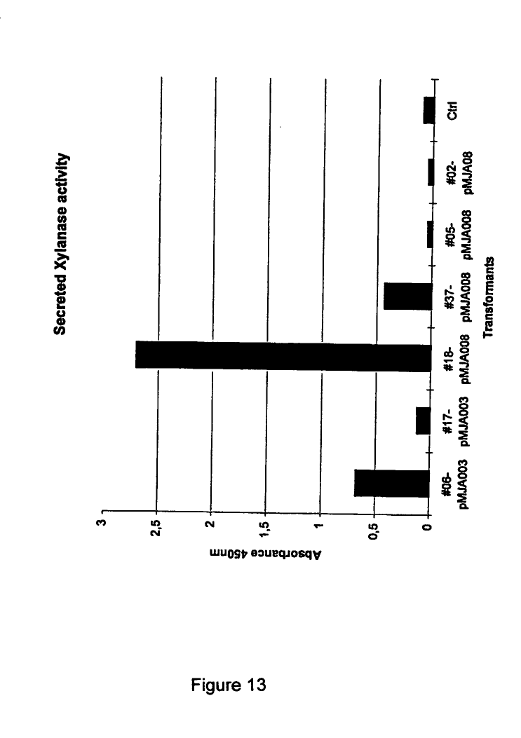

The xylanase activities of the P. funiculosum transformants secreted to the media after 5 days at 25° C. is shown in FIG. 13.

1. A recombinant P. funiculosum strain comprising at least one expression cassette functional in P. funiculosum that is stably integrated into its genome for the production of homologous or heterologous proteins.

2. A recombinant P. funiculosum strain according to claim 1, wherein the expression cassette comprises, operably linked in the direction of transcription, at least one promoter functional in P. funiculosum, at least one nucleic acid sequence encoding a homologous or heterologous protein and at least one terminator region functional in P. funiculosum.

3. A recombinant P. funiculosum strain according to claim 2, wherein the promoter is a promoter of a P. funiculosum gene.

4. A recombinant P. funiculosum strain according to claim 2, wherein the promoter comprises at least one nucleic acid sequence chosen from:
(a) SEQ ID NO:1
(b) SEQ ID NO:2
(c) SEQ ID NO:3 and
(d) a nucleic acid sequence hybridising to any one of the nucleic acid sequences of (a), (b), and (c).

5. A recombinant P. funiculosum strain according to claim 2, wherein the terminator region is a terminator region of a P. funiculosum gene.

6. A recombinant P. funiculosum strain according to claim 2, wherein the terminator region comprises at least one nucleic acid sequence chosen from:
(a) SEQ ID NO:4
(b) SEQ ID NO:5
(c) SEQ ID NO:6 and
(d) A nucleic acid sequence hybridizing to any one of the nucleic acid sequences of (a), (b), and (c).

7. A recombinant P. funiculosum strain according to claim 2, wherein the promoter and the terminator region are from the same gene.

8. A recombinant P. funiculosum strain according to claim 2, wherein said nucleic acid sequence encoding a homologous protein encodes at least one protein chosen from xylanases, cellulases, β-glucanases, ferulic acid esterases, and any cell-wall degrading enzyme from P. funiculosum.

9. A recombinant P. funiculosum strain according to claim 2, wherein said nucleic acid sequence encoding a heterologous protein encodes at least one protein chosen from a fungal protein, a bacterial protein, a plant protein, and animal protein, and a protein from unidentified origin.

10. A recombinant P. funiculosum strain according to claim 9, wherein fungal protein is chosen from an amidase, a phosphatase, a xylanase, a cellulase, a β-glucanase, a ferulic acid esterase, a pullulanase, a phytase, a mannanase, and a milk-cloating enzyme.

11. A recombinant P. funiculosum strain according to claim 9, wherein bacterial protein is chosen from a β-glucuronidase from Escherichia coli, a green fluorescent protein, a pullulanase, an amidase, a phosphatase, a xylanase, a cellulase, a β-glucanase, a ferulic acid esterase, a phytase, a mannanase, and a milk-cloating enzyme.

12. A recombinant P. funiculosum strain according to claim 9, wherein said plant protein is chosen from a phytase and a chitinase.

13. A recombinant P. funiculosum strain according to claim 9, wherein said animal protein is chymosine.

14. A recombinant P. funiculosum strain according to claim 2, wherein the expression cassette further comprises a signal sequence and a pro-sequence inserted between the promoter and the nucleic acid sequence encoding a homologous or heterologous protein, said signal sequence directing secretion of the expressed protein into a culture medium.

15. A recombinant P. funicuiosum strain according to claim 14, wherein said signal sequence and pro-sequence are from a P. funiculosum gene.

16. A recombinant P. funiculosum strain according to claim 15, wherein said signal sequence and pro-sequence are those of the Acidic Aspartyl Protease Apf gene of P. funiculosum, said signal sequence and pro-sequence being represented by SEQ ID NO:7, or by nucleic acid sequences hybridising to said sequence.

17. A recombinant P. funiculosum strain according to claim 15, wherein said signal sequence is that of the csl31 gene of P. funiculosum, said signal sequence being represented by SEQ ID NO:8, SEQ ID NO:9, or by nucleic acid sequences hybridising to said sequences.

18. A recombinant P. funiculosum strain according to claim 1, further comprising an expression cassette comprising at least one nucleic acid sequence encoding a dominant selection marker.

19. A recombinant P. funiculosum strain according to claim 18, wherein the dominant selection marker is an enzyme degrading at least one compound chosen from a compound with an antibiotic activity and a compound with a fungicide activity.

20. A recombinant P. funiculosum strain according to claim 19, wherein the compound with antibiotic activity is chosen from hygromycin, kanamycin, oligomycin, streptothricin, and phleomycin.

21. A recombinant P. funiculosum strain according to claim 19, wherein the compound with fungicide activity is chosen from benomyl and biaiophos.

22. A recombinant P. funiculosum strain according to claim 1, further comprising an expression cassette comprisinq at least one nucleic acid sequence encoding a protein capable of complementing an auxotrophic P. funiculosum to prototrophy.

23. A recombinant P. funiculosum strain according to claim 22, wherein the nucleic acid sequence encoding a protein capable of complementing an auxotrophic P. funiculosum to prototrophy encodes an enzyme implied in nucleoside biosynthesis or an enzyme implied in amino-acid biosynthesis.

24. A recombinant P. funiculosum strain according to claim 22, wherein the nucleic acid sequence encoding a protein capable of complementing an auxotrophic P. funiculosum to prototrophy is chosen from pyrA, pyrB, pyrG, pyr4, argB, and arg4.

25. A recombinant P. funiculosum strain according to claim 22, wherein the nucleic acid sequence encoding a protein capable of complementing an auxotrophic P. funiculosum to prototrophy is trpC.

26. A recombinant P. funiculosum strain according to claim 22, wherein the nucleic acid sequence encoding a protein capable of complementing an auxotrophic P. funiculosum to prototrophy is a nucleic acid sequence encoding eukaryotic molybdopterin synthase.

27. A recombinant P. funiculosum strain according to claim 1, further comprising an expression cassette comprising an amdS gene.

28. An expression cassette comprising, operably linked in the direction of transcription, at least one promoter functional in P. funiculosum, at least one nucleic acid sequence encoding a homologous or heterologous protein and at least one terminator region functional in P. funiculosum.

29. An expression cassette according to claim 28, wherein the promoter is a promoter of a P. funiculosum gene.

30. An expression cassette according to claim 28, wherein the promoter comprises at least one nucleic acid sequence chosen from:
(a) SEQ ID NO:1
(b) SEQ ID NO:2
(c) SEQ ID NO:3 and
(d) a nucleic acid sequence hybridising to any one of the nucleic acid sequences of (a), (b), and (c).

31. An expression cassette according to claim 28, wherein the terminator region is a terminator region of a P. funiculosum gene.

32. An expression cassette according to claims 28 or 31, wherein the terminator region comprises at least one nucleic acid sequence chosen from:
(a) SEQ ID NO:4
(b) SEQ ID NO:5
(c) SEQ ID NO:6 and
(d) a nucleic acid sequence hybridising to any one of the nucleic acid sequences of (a), (b), and (c).

33. An expression cassette according to claim 28, wherein the promoter and the terminator region are from the same gene.

34. An expression cassette according to claim 28, wherein said nucleic acid sequence encoding a homologous protein encodes at least one protein chosen from xylanases, cellulase, β-glucanases, ferulic acid esterases and any cell-wall degrading enzyme from P. funiculosum.

35. An expression cassette according to claim 28, wherein said nucleic acid sequence encoding a heterologous protein encodes at least one protein chosen from a fungal protein, a bacterial protein, a plant protein, an animal protein, and a protein from unidentified origin.

36. An expression cassette according to claim 35, wherein said fungal protein is chosen from an amidase, a phosphatase, a xylanase, a cellulase, a β-glucanase, a ferulic acid esterase, a pullulanase, a phytase, a mannanase, and a milk-cloating enzyme.

37. An expression cassette according to claim 35, wherein said bacterial protein is chosen from a β-glucuranidase from Escherischia coli, a green fluorescent protein, a pullulanase, an amidase, a phosphatase, a xylanase, a cellulase, a β-glucanase, a ferulic acid esterase, a phytase, a mannanase, and a milk-cloating enzyme.

38. An expression cassette according to claim 35, wherein said plant protein is chosen from a phytase and a chitinase.

39. An expression cassette according to claim 35, wherein said animal protein is chymosine.

40. An expression cassette according to claim 28, further comprising a signal sequence and a pro-sequence inserted between the promoter and the nucleic acid sequence encoding a homologous or heterologous protein, said signal sequence directing secretion of the expressed protein into a culture medium.

41. An expression cassette according to claim 40, wherein said signal sequence and pro-sequence are from a P. funiculosum gene.

42. An expression cassette according to claim 41 wherein said signal sequence and pro-sequence are those of the Acidic Aspartyl Protease Apf gene of P. funiculosum, said signal sequence and pro-sequence being represented by SEQ ID NO: 7, or by nucleic acid sequences hybridizing to said sequence.

43. An expression cassette according to claim 41, wherein said signal sequence is that of the csl31 gene of P. funiculosum, said signal sequence being represented by SEQ ID NO:8, SEQ ID NO:9, or by nucleic acid sequences hybridising to said sequences.

44. An expression cassette according to claim 28, wherein said nucleic acid sequence encoding a homologous or heterologous protein encodes a dominant selection marker.

45. An expression cassette according to claim 44, wherein the dominant selection marker is an enzyme degrading at least one compound chosen from a compound with an antibiotic activity and a compound with a fungicide activity.

46. An expression cassette according to claim 45, wherein the compound with antibiotic activity is chosen from hygromycin, kanamycin, oligomycin, streptothricin, and phleomycin.

47. An expression cassette according to claim 28, wherein said nucleic acid sequence encoding a homologous or heterologous protein encodes a protein capable of complementing an auxotrophic P. funiculosum to prototrophy.

48. An expression cassette according to claim 47, wherein the nucleic acid sequence encoding a protein capable to complementing an auxotrophic P. funiculosum to prototrophy encodes an enzyme implied in nucleoside biosynthesis or an enzyme implied in amino-acid biosynthesis.

49. An expression cassette according to claim 47, wherein the nucleic acid sequence encoding a protein capable of complementing an auxotrophic P. funiculosum to prototrophy is chosen from pyrA, pyrB, pyrG, pyr4, argB, and arg4.

50. An expression cassette according to claim 47, wherein the nucleic acid sequence encoding a protein capable of complementing an auxotrophic P. funiculosum to prototrophy is trpC.

51. An expression cassette according to claim 47, wherein the nucleic acid sequence encoding a protein capable of complementing an auxotrophic P. funiculosum to prototrophy is a nucleic acid sequence encoding eukaryotic molybdopterin synthase.

52. A vector comprising an expression cassette according to claim 28.

53. A vector according to claim 52, which is a plasmid, a linear DNA, a phage or a virus.

54. A host cell comprising a vector according to claim 52.

55. A host cell according to claim 54, which is a fungal cell, a bacterial cell, a plant cell, or an animal cell.

56. A host cell according to claim 55, wherein the fungal cell is a P. funiculosum cell.

57. A method for producing recombinant P. funiculosum comprising:
(a) generating protoplasts of P. funiculosum,
(b) co-transforming, in the presence of PEG, the protoplasts generated in (a) with at least two vectors
according to claim 52, one vector containing the expression cassette encoding a dominant selection marker and a second vector containing the expression cassette,
(c) selecting recombinant P. funiculosum transformed in (b) with a selection agent corresponding to the dominant selection marker.
(d) recovering recombinant P. funiculosum containing the expression cassette the first vector and the expression cassette of the second vector stably integrated into their genome.

58. A method for producing recombinant P. funiculosum comprising:
(a) generating protoplasts of P. funiculosum,
(b) co-transforming, in the presence of PEG, the protoplasts generated in (a) with at least two vectors according to claim 52, one vector containing the expression cassette encoding a protein capable of complementing an auxotropic P. funiculosum to prototrophy and a second vector containing the expression cassette,
(c) selecting recombinant P. funiculosum transformed in (b) with culture conditions corresponding to said prototrophy.
(d) recovering recombinant P. funiculosum containing the expression cassette of the first vector and the expression cassette of the second vector stably integrated into their genome.

59. A method for producing recombinant P. funiculosum comprising:
(a) generating protoplasts of P. funiculosum,
(b) co-transforming, in the presence of PEG, the protoplasts generated in (a) with at least two vectors according to claim 52, one vector containing the expression cassette comprising the amdS gene and a second vector containing the expression cassette,
(c) selecting recombinant P. funiculosum transformed in (b) with culture medium containing acetamide as single N-source.
(d) recovering recombinant P. funiculosum containing the expression cassette of the first vector and the expression cassette of the second vector stably integrated into their genome.

60. A method for producing recombinant P. funiculosum comprising:
(a) generating protoplasts of P. funiculosum,
(b) transforming, in the presence of PEG, the protoplasts generated in (a) with a vector according to
claim 52, containing both the expression cassette encoding a dominant selection marker,
(c) selecting recombinant P. funiculosum transformed in (b) with a selection agent corresponding to the dominant selection marker.
(d) recovering recombinant P. funiculosum containing both expression cassettes stably integrated into their genome.

61. A method for producing recombinant P. funiculosum comprising:
(a) generating protoplasts of P. funiculosum,
(b) transforming, in the presence of PEG, the protoplasts generated in (a) with a vector according to
claim 52, containing both the expression cassette encoding a protein capable of complementing an auxotrophic P. funiculosum to prototrophy and the expression cassette,
(c) selecting recombinant P. funiculosum transformed in (b) with culture conditions corresponding to said prototrophy.
(d) recovering recombinant P. funiculosum containing both expression cassettes stably integrated into their genome.

62. A method for producing recombinant P. funiculosum comprising:
(a) generating protoplasts of P. funiculosum,
(b) transforming, in the presence of PEG, the protoplasts generated in (a) with a vector according to
claim 52, containing both the expression cassette comprising the amdS gene and the expression cassette,
(c) selecting recombinant P. funiculosum transformed in (b) with culture medium containing acetamide as single N-source.
(d) recovering recombinant P. funiculosum containing both expression cassettes stably integrated into their genome.


SEQUENCE LISTING (37 sequences)

SEQ ID NO: 1 - DNA, 452 bp, Penicillium funiculosum
actagtgagtatgtaagcgagtctgatcttcccgcccgccaatcaaatcgcaggatcacagaatgcaaataccttctgatcccaaccacaaccagtatttctgattcgaatcaaaatcgtggcatgtaattcgcgggatcagaatgcggtttccaccggccaaaatgaccgatctgcgttgccacggctggtaatggcaggatcagagcagcagcgttgaatcagtcagatcaagcggcgctcccgctgattggccagatcacccactagcgcgtgatcccaggcaccttcccgcgtccggctctttctatagaaatatctcccgccgcgtcttccttcccaccttctttctcctctccatctccagctccatcttcaagcttactcaacattatcaacgcgtttacgcatccacattcaattacttatcatctcatccccatacagaattc

SEQ ID NO: 2 - DNA, 2213 bp, Penicillium funiculosum
  Promoter 10: Promoter Region from Acidic Aspartyl Protease gene wherein n is any nucleotide
gaattcttgntcacctcctgtacccatcctcgacttgtcctgcatagcaagtctacagagacagtgcctgtgaattcgcatgctaaaaacgcaatacaggttatcatctatgccagggtgagggggcttctttctcattctatccctagctggtgctagcgctcaaatcctccaatagccctactcctaacccaacttatcctggacatcctggattgttcatcgaacttctgtctttttactatcgcatcccgcggtgacctcgggatattgactacagatatgatcacttgacagctgcacgaaatatcattggccactgccgtgatagataactagctcgctaaacacccatgtacggcctagtggatacacacggttattgaaggatcaagtcaagagccggcgatctgacaacttgacaacttggacttttggaatttggaggcttgcttgcagtcgcatcaaattaccgggctctccgggattcaacgctcggaagttgctactcgctacaacctgtctaggtgaacgccgtaaacagttagttgtgcttctatcccaacaaatggcttgatgctattggactcatgccaacagactcaaaattagtacgggtggagtcatcttcgcgcgatcatacctaacatcttgactagtgccgccagctgggacttagccgtagatctctcgtttcttatcattcttcatgaccttgattggattttggcgatattgacatcggatgcccacgggctttgcattgatggcatgtatcaatgcatgtacggacgtacaaggcaaacattttcgaaacatcatatcgagcttccttttccgaaagtcaagggatgcgctgtcgcatcgataaccaacaagcccccaccttcacatggtaagaaatcgaccttaatgcaacacaattggttccatgcgatctgataagttgtatctgttaatgactcgacggagacctccgagagcctcattcgatgatccaagcagcctgattgccacggattaagattaaagctgtttcacggtttcgcggccgaggctttctacaacgtgccactataatcattagcaagacatgccgagctgcgtctcgtatcgttggtcatcgccgtgcctccaaagcgatcacaatcttcacgatgtatgtgcatgactagatgaataggaggtaaaccacacgtgttgagaaaagtcgaccatcaatcctgcattcatacatggcaatgttgtattgcttaaatcatgacacccataggaagcatccttcggcataatactatcctccgtgccctgatcgttgcccgcacaagcattgctttttataaaaatggaacctgagcctcaacgcagatcgcaaatcagatccacccgtcgagccatgttcagcaactcccccgtcaggctcagtgtgacctaccacatcgaggctaaaccggtggagtcgaatacggggttgaattttctagattgctacctactttacgagccgccaccaaccagggctagtcgttggcgatgatcgttgagctcgggatttaggaacaaggctaatgctagatgggctgaggcttatgcatgatcaagcattggacggtagcaatcgtgggatctgccggactaaaaacatcgctgactaggcataggtccgtgggaaggcatttgctttttgaaacctcaatgcgatggacaagccactgccaggcaagatctgccatttggttcaatgctttcaagcgacagcaagcctattctcagctcccattcggcacgtcattgaacatgacaataacgaatccaacgtgattcgctcggctgtttattgtttctagattgctcctacctgatttggactgtttccaaagatacgacatactacaatggggctctgatcagacaagcatccgccattaccccctcttcgtatgtcggcgatgtttcttatctcggccaaacgttttatgatcgacggcgtaatgtttcgttgtcagaagcaccacagaccagagaggaatcacggaagaaaagatgcgacatcgttatcttcgtcgattcatacgtgatgactgatatgggattactttggtgatcaaaagtataaatatcgagtcaagttcactcgatcacttggctttcttctcatcatcatcgtcacaatcattcactctcatatcaatctttcgggctatctcttcaac

SEQ ID NO: 3 - DNA, 1351 bp, Penicillium funiculosum
gcatgcccactccaatgactgattcattagatgagtttctgcacaatagccaagagtctatgagctgatcagtagcattttccccagtcatcacgtctgtcgttgctgcagtcattgacatgtaatccggacgaggcatttttaaacccggttagaacgatttgaagacgcacgcctcctcacaactacgtcgcacagaactaaaaattatcagactctgaatgatcatgtgggaagtatccttgtgctgcggttaaatgcacttgcataggcatgaatatggcttcattttactctggtcgacgcaatgtaactacgtacccggattatccgtacaaccaacccctgcaaaaagagcgagaaggcctatataacacactttcgcttttccatagaatcatacaccgagataatgttaccatgggcagccgtcacccctgagacaatgaaaagtattctctacatccttctagggacagaatataatatagctacctaggaccatttggggctaaatcaaagtgtctccaccaaccataataagcatatctgcagaaacgaattggctcacttgttggagacccaaagtagcagatcatgtgtaacagacgccatcgccgagactaatgatgaaccacaaacatgccatgtccacgttcctgtcgatctagagctgtctatcatgcaattcgccttttgcttgctccactctcaacatggattataagacacactgactttttgaagctgtaagcctaggtcttgacgatggcaaaatgtgatattttgcatcgatgtttgaatgaactcagattttcgttcggcgtcaatccgtacctcagctcttcttgtcacaacttgggtcctgtctcttgcatcaaggtttgaatgaactcggattttcgttcggtgtcaatctggatttcagctctcatcttgtcacaattttgagtcctgtatcctaacgcaagccacgctgaaagtcgcgtctgtattattgtttgtagccaccaagtatgactctcaaaggcctagaactgcagaccaagtctttcgtcggccaaacttccagaaacaagaaaacttttacataaaagaattgaggaattggataatacatcaagaccaacccacatggacagtcgccattgaacgaggagacgtaggtaaacattgcccttggatcatgggttgaggtaacattgtttaaaagggtaaccagttgcgaattctggccttttcaacgccgacaaaaagtataaataaggacgttgagctggtacctcgtccgtaatctgatcctctatcaatagtatcttcaacctcaatattcagtagaaaaaacaacata

SEQ ID NO: 4 - DNA, 314 bp, Penicillium funiculosum
ctcgagcgttttctcttcattgttttgtcgcggcctcgccaacgcggtcgacgcgtctgctcgccgctgtggtgacgcgttgctccttattctgggttgggtttcggtatgacagggtcgtcagggtgttctctgaatagtttttcttgttttcttcacgtctttatgggtctgggtcttgcatctttttactactacttaaattatacctcgtctacactggtcggcttgtggctatggtccgaatatctttgtatcatagcgcgttatcctgtgactgagttgcgactgctcgtcatcgcacatgaggtacc

SEQ ID NO: 5 - DNA, 2198 bp, Penicillium funiculosum
cttaatcattctcaagggattttgacctgaaaagtcaacattgaaatgattgtgattggattatgattgaacaaaattgttaatattcaaaatgatggaatttagtacatatgtacataacccatactgcacatatggaatttctatacttgctgattgtatccttgtaattcataaacactgtatccaactctctaacaaattcaagcctgagccgcaacatctccatcctccctccacgcaaaccacgccaaaacaataaaccccttcaacccaaccgccgtccacagagcgcccatcaaaccaacctcactcctcgcccaaaacccacccatcacagcacccaccaacaaacacacaaccgcaccaagcctcctcctctcctccacatccttactcaatgccatccccgaaacaaaatccggatgagaaaacaaatcacaataaacactcgtcaaaaccacactcgacaaccctcctagttttaacacgcggctgataaccgcttgtcctgagctctgaaatgcgaccatacccaaagggacgatgacgcgccatgttagtccggaagtttttgagggtcgttcgagtgtgacgattatcgcggcacatgttatgcaggccatttggagggcgaaactagccattagaacccatctcttcctcggtgaaaaggatcggtggaaggcgccgaatagtaacgagccgagacagaagctggcaatcgaaataccggatttgatccagcggtcgtcgttaccaccattaccaccatcacgaccattacgaccgccgttgcctgaactatggtggcttatttgggatgctagtcctaggccgagatagactgtgttgccggtttgcatgctcacgaaggagccccagatgaatactgcggagctgtcgagcaggccggtgataatgtagcagtatagcagaaccaggtcggtgtggtgcgttgaaaggggggcgttgaggtggtgccgtagtcttttgaagccatcgcgttggctactatggctgctattgtgtggtagtaaagggtccgtttcgcgcagttgagtcatgttgccatacagcgcaagaagtggtaaaatatctagaagactagcatatataccagtgcgaaatgtttaagtcggcccataatgatcccttgcctacattccggttgtacatgtacttcgcttccggattgagcttcagcccacatttcgggctgacgtcaacgatgcgacagattctaagacaattgtgcgacttatcccttacttatattattagacggtgacacgagtgaaattatttacgcgattgttctcctatatctaggtacctaggtacggattctagttgtgatatatatgtacggcccgtcaaccaccaatcgatctgccaggaagtacccagacaatcaaacgagccgtcaatacaatgcataaaccaatcctcctaggcctaggattgctttcagtccttaccaacgccttcccagtcccagcagaaaacttccaacgtctatccagaatcccaccatcttcatctcagcaatactttggcgacatccatgatggcaagcacacgaacaaaccatttacacccgaccatcgagacccttacgatcgcaaagtcgatcccatccccgacaaacttgagccattaccattccgcaacggccacggagcaacggtcatgggtccccgaaataaggaccgcgaacgacagaatccggatctcgtgaggccgccgagtacggatcatgggacgttgagtaatatgcgatggagttttgcggattcgcatatcagaattgaggtaacccctttctctgctctgaggaaatctccgaaggaggagattcagtactgatagacatggaaggaaggaggatgggctcggcaaaccaccatccgcgaactacccaccagcaaagaactagccggcgtcaacatgcgtcttgaggcaggtgcaattcgcgaattgcactggcatactgaagccgaatgggcgtttatgctagctggcaaagcccgtgtcaccggactagatacggaggggggaagtttcatcgatgatgtcgaagcaggtgatctgtggtactggcctgctggtcatccgcactcgattcaagggttgggtccgaatggcactgagtttttgttgatttttgacgatgggaatttttcggaggagtcgac

SEQ ID NO: 6 - DNA, 1211 bp, Penicillium funiculosum
tagacagatatatgtcgacagtatatcacggggaaggccgcttttgttgtgtgacaaataggagagatatttcatgtttgagctttagatctgttagcttgcatcaataacaatatcaggagagggagtgcaatacgaaattcatatttcgagacaattgtatgttcaactatcattagcagggcctgactaggatctgcttataagatacttcagcatcctatatttacgcttagagcagtttcttgttttagtagtgatttgtttgacagttgttgaatttatttattttgctttattttctgccttgcttgagaaaaaggcattaatttgagaagtgccgagccaatcggcattccattcaggtctcgcatcctggcctgagcgcgcgcctcaccagtgactggtggtttttatcgtggcgtacggtatacaagcccgctgtcagcttatcccgattagttaatttgaggcgagcggcggttacgatcataagcactcagccacatccgcaacgacaataaataggaatgcgaaatggcccaagaatccagttcttttgtcttcagctatatctgtcaacatgtccattgaaacaagtgcagcagccaacggcgcccacggggccaaagacgagatcctagtacacccgtgccacccacagaggctggagagatgctaacgcaatagcaacaggtcctttctcgatgcgtcaatccagaaggaatctcctggccagccatctggtatttccccaccagtgccccttcattctcgatatgttcctattgctatgacaaacacatccaacccacgcctcatgaaggccttttcgaggctctctgggcagacgggagttttcgcataacctgttactggaatactccgcgggtgatgtcgcatctggccgctgatgactgggatgcgatcaacgccttcatggtcgagcggaccaaaatcccagtctgcaaagggcaggcaggtcacaccggacttgatggctcccgttggtatggcatgatcggcgcatgggagattcagggcttcgtgatatgcgaagcgtgctattacgatctggtggtgtggaatgagcttaggacgtacttcaccacgacgccgacaatcaagtcggaggagggaatatggacatgcgatgcggcggtgccactaataaacgagggactccgtcgggcgatcacgtcacgcaacag

SEQ ID NO: 7 - DNA, 210 bp, Penicillium funiculosum
  CDS (1)..(210)
  sig_peptide (1)..(57)
atggttaactccaagactgtcgtctctgccttggccctttcggcccttgctgcagctgcccctgcccctgcccccagcaagaccaccagcttctccatcagccaggttgctgtcaagaagcctttggtccaccccgctgccaaatatgccaaggctcttgccaagttcggtgctgaggtccctgctcaagttgcctctgctgctgcctcc

SEQ ID NO: 8 - DNA, 66 bp, Penicillium funiculosum
  CDS (1)..(66)
  sig_peptide (1)..(66)
atgcagaccaaaatctttttttctccccttctccaagccttctggctctcatcagcacttgcagcc

SEQ ID NO: 9 - DNA, 66 bp, Penicillium funiculosum
  CDS (1)..(66)
  sig_peptide (1)..(66)
atgcagaccaaaatctttttttctccccttctccaagccttctggctctcatcagcacttgcaccc

SEQ ID NO: 10 - DNA, 20 bp, Artificial Sequence
  Oligonucleotide NB063
gcyaagcgycaymghaagat

SEQ ID NO: 11 - DNA, 20 bp, Artificial Sequence
  Oligonucleotide NB064
rccgaarccgtacmrrgtdc

SEQ ID NO: 12 - DNA, 26 bp, Artificial Sequence
  Oligonucleotide NB156
gaattcactagtgagtatgtaagcga

SEQ ID NO: 13 - DNA, 24 bp, Artificial Sequence
  Oligonucleotide NB157
gaattctgtatggggatgagatga

SEQ ID NO: 14 - DNA, 30 bp, Artificial Sequence
  Oligonucleotide MJA004
ccgctcgagacgttttctcttcattgtttt

SEQ ID NO: 15 - DNA, 26 bp, Artificial Sequence
  Oligonucleotide MJA005
ggggtacctcatgtgcgatgacgagc

SEQ ID NO: 16 - DNA, 31 bp, Artificial Sequence
  Oligonucleotide PEP5′
gacttygacactggctctgcygatctgtaag

SEQ ID NO: 17 - DNA, 28 bp, Artificial Sequence
  Oligonucleotide PEP3′
gagcaggaggagrgtggtaccggtrtct

SEQ ID NO: 18 - protein, 15 aa, Penicillium funiculosum
  UNSURE (1)..(2): N-terminal peptide from protein product of Csl31 gene wherein Xaa is any amino acid
XXYQTRIFEAGTTFG

SEQ ID NO: 19 - DNA, 48 bp, Artificial Sequence
  Oligonucleotide QT
ccagtgagcagagtgacgaggactcgaactcaagcttttttttttttt

SEQ ID NO: 20 - DNA, 18 bp, Artificial Sequence
  Oligonucleotide Qo
ccagtgagcagagtgacg

SEQ ID NO: 21 - DNA, 17 bp, Artificial Sequence
  Oligonucleotide GSP12
ccagacccgcatcttcg

SEQ ID NO: 22 - DNA, 18 bp, Artificial Sequence
  Oligonucleotide Qi
gaggactcgagctcaagc

SEQ ID NO: 23 - DNA, 15 bp, Artificial Sequence
  Oligonucleotide GSP22
gaggccggcaccacc

SEQ ID NO: 24 - DNA, 18 bp, Artificial Sequence
  primer_bind 4: redundant Oligonucleotide Furniss 3 wherein n is any nucleotide
ccanrmrttraartgrtt

SEQ ID NO: 25 - DNA, 18 bp, Artificial Sequence
  primer_bind 3,6,9,14: redundnat Oligonucleotide Furniss 5 wherein n is any nucleotide
ytngcngtntayggntgg

SEQ ID NO: 26 - DNA, 32 bp, Artificial Sequence
  Oligonucleotide MJA001
cggaattcaaaatgaagctcttcctagctgca

SEQ ID NO: 27 - DNA, 29 bp, Artificial Sequence
  Oligonucleotide MJA002
ccgctcgagctggaggtgtcacagtatct

SEQ ID NO: 28 - DNA, 30 bp, Artificial Sequence
  Oligonucleotide MJA003
ccgctcgagctaggacactgtgatggtact

SEQ ID NO: 29 - DNA, 20 bp, Artificial Sequence
  Oligonucleotide NB116
gacatgctatcagagctgag

SEQ ID NO: 30 - DNA, 27 bp, Artificial Sequence
  Oligonucleotide NB117
gtccagtcattttgatagatgtatggg

SEQ ID NO: 31 - DNA, 27 bp, Artificial Sequence
  Oligonucleotide NB118
acgttttctcttcattgttttgtcgcg

SEQ ID NO: 32 - DNA, 37 bp, Artificial Sequence
  Oligonucleotide ApfA
gtagccatggaggcagcagcagaggcaacttgagcag

SEQ ID NO: 33 - DNA, 49 bp, Artificial Sequence
  Oligonucleotide ApfB
gtagccatggcttaatcattctcaagggattttgacctgaaaagtcaac

SEQ ID NO: 34 - DNA, 32 bp, Artificial Sequence
  Oligonucleotide CslDel1
tatagccggctgcaagtgctgatgagagccag

SEQ ID NO: 35 - DNA, 41 bp, Artificial Sequence
  Oligonucleotide CslDel2
agatgccggctagacagatatatgtcgacagtatatcacgg

SEQ ID NO: 36 - DNA, 27 bp, Artificial Sequence
  Oligonucleotide Csl12
aatgttaccatgggcagccgtcacccc

SEQ ID NO: 37 - DNA, 32 bp, Artificial Sequence
  Oligonucleotide Csl13
tcactagtgattatacccgggtgcaagtgctg